(19) United States
(12) Patent Application Publication
(10) Pub. No.: US 2004/0248189 A1
(43) Pub. Date: Dec. 9, 2004

(54) Method of making a library of phylogenetically related sequences

(76) Inventors: Grzegorz Bulaj, Salt Lake City, UT (US); Baldomero M. Olivera, Salt Lake City, UT (US)

Correspondence Address: TRASK BRITT, P.O. BOX 2550, SALT LAKE CITY, UT, 84110, US

(21) Appl. No.: 10/456,375

(22) Filed: Jun. 5, 2003

(51) Int. Cl. G01N 33/53; G06F 19/00; G01N 33/48; G01N 33/50

(52) U.S. Cl. 435/7.1; 702/19

(57) ABSTRACT

The invention includes a method of assaying phylogenetically related sequences and generating a set of possible nucleic acid or amino acid sequence combinations. Sequences are analyzed to create an alignment and a set of phylogenetically related sequences are selected. The observed residue(s) or indel(s) occupying each position in the alignment of the selected phylogenetically related sequences are identified and this information is used to generate a set of sequence combinations, wherein the set of possible sequence combinations (nucleic acid or polypeptide) is composed of the union of the observed residues or indels identified at each position.

### TECHNICAL FIELD

The invention relates to biotechnology generally and more particularly to a method of preparing a library of nucleic acid or amino acid sequences and the resulting libraries.

### BACKGROUND

As the amount of information available to biologists increases, data analysis and productive use of the information becomes progressively more challenging. Sequence homology has been a very versatile tool that can be employed to assist in numerous tasks, from establishing the function of a gene to determination of the evolutionary development of an organism. Numerous specialized tools have been established in the public domain, which serve to align homologous sequences.

With the steady increase in sequence data, more homologies between related sequences, either at the nucleic acid level or the amino acid level, are being identified. As the number of homologous sequences increases, a more comprehensive phylogenetic comparison, both across species and within species, becomes possible.

Traditionally, sequence alignments have been used to identify conserved regions (i.e., those regions of a nucleic acid or protein where all members of the alignment have the same nucleotide or amino acid). More recently, conservative substitutions, non-identical nucleotides or amino acids, have been identified and included with the analysis of conserved regions.

The traditional view is that conserved sequences, which may or may not include conservative substitutions, have a function that is subject to strong evolutionary selection. This function is often presumed to be maintained both within a species and across species. For example, kinase domains have been identified in numerous protein families and these domains are presumed to retain the function of phosphorylation. See, Doray et al. (2002). Even in the absence of biochemical data, sequences, such as proteins, may be presumed to have a particular function based solely on homology to sequences having a known function. See, Stoilov et al. (2002).

The premise of retained function is most frequently applied in reverse, where new members of a protein family are isolated from other species based solely on their sequence homology. Therefore, the identification of homology between two or more proteins and/or nucleic acids facilitates the ability to isolate related sequences from new species. However, homology alone does not predict the precise biochemical function of the homologous sequence or region.

Conserved amino acid positions may be viewed as those positions which generally may not be altered without loss or reduction of biological activity. Not withstanding the foregoing, conservative substitutions may be made in conserved positions. Conversely, non-conserved positions are traditionally viewed as positions lacking a clear or necessary role in the biological function of the protein. As a result, assays directed at identifying the function of a domain traditionally ignore, or only tangentially address, the biological role of the non-conserved positions.

The analysis of conservation in nucleic acid sequences depends on the function of the sequence. Nucleic acid sequences, which, for example, serve as binding sites for transcription factors, or other similar functions, such as t-RNAs and ribozymes, are expected to show conservation at the nucleotide level. Nucleic acid sequences, which encode a protein, may be considered to be homologous when the non-conserved nucleotides do not change the encoded amino acid. Thus, due to the degeneracy of the genetic code, silent mutations or changes in the coding sequence may be ignored when considering the effect on the gene product.

When sequence homology identifies conserved positions in multiple sequences and where the function of the conserved positions is unknown, the presence of conservation between sequences may be used to infer the presence of a functional domain. However, the function of the presumed domain must still be determined.

Identifying a function for a molecular domain has traditionally been done by studying the function of an individual member sequence and then imputing that function to the other members of the family. However, this approach suffers from the limitations imposed by the use of a single member sequence. For example, an individual member sequence may not be compatible with a particular assay used to determine function. For example, a potential family of ligands, having unknown function, is traditionally tested using an individual member ligand and assaying for binding to a class of receptors. In this case, an individual member may exhibit a binding preference inconsistent with the particular receptor source (e.g., mouse versus human) used in the assay and prevent the identification of function. Alternatively, the function of a family of ligands may be screened using a combinatorial approach. In this case, a library may be generated and screened for binding to a class of receptors. Under this approach, the library is generated by holding constant those positions having identity and randomizing non-conserved positions. This approach requires the screening of a large number of sequences. This combinatorial approach suffers from a number of limitations.

The invention overcomes the limitations of the individual member and combinatorial approaches.

### SUMMARY OF THE INVENTION

The invention includes a method of identifying conserved and non-conserved substitutions within a set of sequences, wherein one or more non-conserved substitution exhibits desired properties. The invention further relates to a method of assaying phylogenetically related sequences including conservative substitutions.

The amino acid and nucleic acid sequences of the invention interact with specific molecules. For example, amino acid sequences which specifically bind to a receptor or a receptor subtype or nucleic acid sequences that specifically bind to a ligand binding molecule. The invention further relates to nucleic acids that encode polypeptide sequences.

The invention provides an alignment of phylogenetically related sequences integrated with methods of generating a set of sequences based on the composition of the phylogenetically related sequences. The set of sequences (a library or a cladistic library) utilizes the members observed at each position, conserved positions and allowed substitutions (conservative substitutions and non-conservative substitutions, including indels), to reduce the complexity of a library. The members observed at each position of the sequence alignment are identified and from this information a set of sequence combinations composed of the union of observed members at each position is generated. Thus, the number of sequences which must be generated is reduced. Furthermore, a library or set of sequences prepared by the methods of the invention extends the possible benefits conferred by each allowed substitution to combinations not present in the natural sequences.

To further reduce the number of sequences, conservative substitutions occupying a position may be treated as being identical to a single residue occupying that position. Accordingly, a representative of the conservative substitutions is selected and the conservative substitutions at that position are deemed to be equivalent to the chosen representative. The choice of residue deemed to represent the members of the conservative substitutions may be selected based on the frequency of occurrence in the sequence alignment or on other criteria known in the art.

The invention further relates to sequences (peptides and/or nucleic acids) identified from sets of sequences generated by the methods disclosed herein. When the sequence is a polypeptide, such as a conopeptide, the identified sequence specifically binds to a target receptor. The invention also relates to nucleic acids which encode peptides, for example, nucleic acids encoding peptides that specifically bind to a receptor. In addition, the invention relates to nucleic acid sequences having a function other than encoding a peptide, for example, ribozymes, promoter elements, regulatory elements, splicing signals, polyadenylation signals and tRNAs.

The invention relates to a method of generating a set of possible amino acid sequence combinations, wherein the amino acid sequences are analyzed to create an alignment, and a set of phylogenetically related sequences are selected. The observed amino acid residue(s) or indel(s) occupying each position in the alignment of the selected phylogenetically related sequences are identified and used to generate a set of possible amino acid sequence combinations, wherein the sequence combinations are composed of the union of the observed amino acid residues or indels identified at each position.

The invention relates to a method of generating a set of possible nucleic acid sequence combinations, wherein nucleic acid sequences are analyzed to create an alignment, and a set of phylogenetically related sequences are selected from the analyzed sequences. The observed nucleic acid residue(s) or indel(s) occupying each position in the alignment of the selected phylogenetically related sequences are identified and used to generate a set of possible nucleic acid sequence combinations, wherein the sequence combinations are composed of the union of the observed nucleic acid residue or indels identified at each position.

The invention further relates to a method of generating a set of possible nucleic acid sequence combinations, wherein amino acid sequences are analyzed to create an alignment and a set of phylogenetically related sequences are selected. The observed amino acid residue(s) or indel(s) occupying each position in the alignment of the selected phylogenetically related sequences are identified and used to generate a set of possible nucleic acid sequence combinations, wherein the nucleic acid sequence combinations encode a set of polypeptides composed of the union of the observed amino acid residues or indels identified at each position.

The invention also relates to screening or selecting a set of possible amino acid or nucleic acid sequence combinations to identify ligand binding pair members and isolating an identified individual ligand binding pair member or a mixed population of identified individual ligand binding pair members. Further, the identified ligand binding pair member may be produced by chemical synthesis or produced in a recombinant host. A set of possible amino acid or nucleic acid sequence combinations may be screened using phage display, arrays or other ligand presentation systems.

The invention also relates to a set of sequences, wherein the set of sequences is the union of observed members at each position of an alignment.

In another aspect of the invention, the phylogenetically related sequences have a functional relationship (for example, λ-, ω-, σ-, κ-, χ-, τ-, α- and/or α-conopeptides), wherein the sequences form a clade or a part of a clade.

The invention further relates to computer programs which execute the methods of the invention.

The invention also relates to sets of sequences (amino acid or nucleic acid) wherein individual sequences of the set occupy known and/or isolated locations, for example, microarrays, biochips or chips.

### BRIEF DESCRIPTION OF THE DRAWINGS

FIG. 1 shows the phylogenetic relationship of numerous conotoxin sequences from the extensive Cognetix conotoxin database. Six of the amino acid sequences are illustrated along with their relationship to other sequences, which correspond to SEQ ID NOs:1, 2, 3, 4, 5 and 6, respectively.

FIG. 2 shows the six amino acid sequences illustrated in FIG. 1 and the phylogenetic relationship of the sequences.

### DETAILED DESCRIPTION OF THE INVENTION

Conopeptides, which constitute a large source of phylogenetically related sequences of the invention, are small gene products (8-60, more typically 10-40 residues) derived from Conus snail venoms, often stabilized by disulfide bonding between highly conserved cysteine residues (Norton and Pallaghy, 1998). Conopeptide precursors are expected to conform to a three-part structure (signal-propeptide-mature). The disulfide bonds of the conopeptides provide a structural scaffold that tolerates high variability in the intercysteine loops. The high degree of variability allows for the targeting of diverse receptors. For example, the “six-cysteine, four-loop” scaffold (C . . . C . . . CC . . . C . . . C) is shared by conopeptides targeting multiple subtypes of voltage-gated sodium, calcium and potassium channels, including three different sites on sodium channels (McIntosh et al., 1999).

Conopeptides are highly selective receptor ligands, which has facilitated their use as pharmacological tools (McIntosh et al., 1999), and resulted in substantial interest in their potential as neuronal drugs (Bowersox, S. S., and R. Luther, 1998). The spacing of cysteine residues appears to be important for productive folding of such peptides (Drakopoulou et al., 1998) and the number of naturally occurring conopeptide scaffolds so far identified is limited. These scaffolds define large hypervariable families that may share a common evolutionary origin (Conticello et al., 2001). Given the diversity of Conus venoms (an estimated 50-200 unique conopeptides per species for a genus of about 500 species), determining the molecular targets and identifying individual peptides with high selectivity for a particular receptor or channel subtype can be difficult and labor intensive. As a result, the structure and function of only a small minority of these peptides have been determined. One representative embodiment of the invention provides a method of identifying conopeptides, using the natural variation of the peptides to identify combinations of allowed substitutions having a desired property. As demonstrated herein, the invention is also applicable to a wide range of sequence alignments.

An overview of conopeptide precursor variability and evolution has been established using an EST strategy to identify conopeptide-encoding transcripts (Conticello et al., 2001). Conus venom systems are ideally suited for such an approach, since conopeptide-encoding transcripts are relatively short (about 0.5 kb) and highly expressed. Sequencing of over 2,000 cDNA clones and PCR products from five different Conus species provided a data set of 170 distinct conopeptide precursor sequences from eight gene families representing three cysteine scaffold superfamilies (Conticello et al., 2001). Numerous conopeptide precursor sequences have now been identified from all eight superfamilies and a multitude of families within each superfamily (Jones et al., 2001). Conopeptide diversity is a reflection of a targeted mutagenic mechanism to generate high variability and subsequent diversifying selection.

Since synonymous substitutions are neutral, the fixation rate can be considered proportional to the mutation rate. Therefore, it can be assumed that the number of synonymous substitutions per synonymous site (Dₛ) (Nei and Gojobori, 1986) is an adequate representation of the mutation rate. Dₛ in the mature peptide region is significantly higher than for the signal domain, with the propeptide region in most families exhibiting an intermediate value. The apparent mutation rates for the mature domain of conopeptides are elevated by about an order of magnitude relative to the signal peptide. Thus, there is hypervariability of intercysteine residues in the mature region of conopeptides.

In addition to hypervariability in the intercysteine loops, positive selection has been reported to play a decisive role in diversification of venom-expressed gene families (Ohno et al., 1998; Duda, T. F., and S. R. Palumbi, 1999). Thus, the hypervariation noted above is believed to be due to strong positive selection and a hypermutation mechanism. One possible driving force for diversifying selection in conopeptides is the prey specialization prevalent in this genus (Kohn, A. J., and J. W. Nybakken, 1975).

As a result of the hypervariation and strong selection in the mature conopeptide, a single amino acid change can have a significant impact on receptor subtype specificity (Luo et al., 1990). For example, a single amino acid substitution is sufficient to alter the receptor subtype selectivity profile by two orders of magnitude (Luo et al., 1990). In addition, an amino acid substitution may result in different biological activity. For example, Wells et al., 1987, showed that exchanging a limited set of amino acids from one protein into another can carry the function of those amino acids into the new chimeric protein. Therefore, the invention utilizes allowed amino acid substitutions observed at each position as a source for new, improved, or altered function, such as targeting molecules or receptors.

In contrast to the hypervariability associated with the intercysteine loops of the mature region, the cysteines, which are necessary for disulfide bond formation and the protein architecture, are very highly conserved. This conservation extends to the individual cysteine codons, TGT and TGC, where there is a strong bias for one or the other codon at different positions in the alignments (Conticello et al. 2001). Since different codons are conserved at different positions, a simple codon bias cannot be responsible, especially in view of the extremely hypervariable environment of the mature domain. Thus, the cysteine organization and/or codon preference may serve as a basis for the assignment of conopeptides to particular superfamilies and families.

The ability to sequence hundreds or thousands of conopeptides genes provides the ability to generate very large phyolgenetic trees. However, this information does not address the molecular biology or the peptide chemistry necessary to ascertain the function of the gene products. The invention combines bioinformatics data and phylogenetic relations to construct a peptide or nucleic acid library that is both practical and provides the necessary ability to address the molecular function of the gene products. In this manner, an effective integration of molecular biology, bioinformatics, and peptide chemistry is provided.

One approach to addressing function involves the generation of a library of peptides where all non-conserved amino acids are randomized, which is referred to as a combinatorial approach (Jeffrey D. McBride et al., 1996). The combinatorial approach requires the generation of very large numbers of peptide sequences. However, peptide synthesis is not easily achievable when the number of peptides to be synthesized is very large. For example, one limitation encountered in peptide synthesis is that, as more peptides are synthesized, the relative concentration of each species declines (the relative concentration of a peptide is diluted by the total number of unique peptide sequences). For example, it has been estimated that a peptide pool having more than about 1×10¹⁴ different sequences will result in only one copy of each sequence per 0.3 μg of peptide (COMBINATORIAL PEPTIDE AND NONPEPTIDE LIBRARIES: A HANDBOOK 238 (Günther Jung ed., 1996)). In addition, a 4.0 μmol peptide synthesis can only generate a limited number of individual sequences. For example, it is estimated that a 4.0 μmol peptide synthesis can generate no more than about 8 ×10¹⁷ individual peptides. Id. Therefore, even where a library of peptides having only a single copy of each sequence is sufficient, the library should contain less than about 8×10¹⁷ individual sequences/synthesis. Since a single copy of any one member is frequently insufficient the number of individual sequences that can be synthesized in any single synthesis reaction decreases rapidly. While multiple synthesis reactions may be conducted and combined, an upper limit of peptide solubility exists, which likewise limits the number of individual sequences that can be screened or selected per unit volume. Furthermore, as the number of unique peptide sequences increases, the ability to properly fold any one sequence is decreased.

As a hypothetical example, an alignment of highly conserved peptides (having over 80% identity at the amino acid level) 23 amino acids in length, would result in a very large number of peptides that must be synthesized when using the combinatorial approach. In this example, approximately 19 amino acid positions would be identical and only 4 positions would be non-conserved. Using the combinatorial approach, the four (4) non-conserved amino acid positions would be randomized. Since there are at least 20 naturally occurring amino acids (newly identified amino acids raise the number of natural amino acids to 21 or 22), randomizing each of the four (4) non-conserved positions requires that (20×20×20×20=160,000) 160,000 different peptide sequences be generated. As this example illustrates, even when the number of non-conserved positions is small, only four (4), the total number of peptides required becomes extremely difficult to generate in a single batch.

Furthermore, of the extreme number of peptides generated using the combinatorial approach, many incorporate deleterious amino acid substitutions. For example, many of the naturally occurring amino acids, when inserted into one of the non-conserved positions, will prevent the function of that sequence. Therefore, the artisan using the combinatorial approach must labor to produce an extreme number of peptide sequences and many of these laboriously produced peptides will not function.

The present invention, allows the generation of a practical number of peptides that retain function. Natural selection functions to eliminate deleterious mutations and introduce function-enhancing changes. In the case of a Conus toxin, for example, a mutation that prevents the function of a toxin reduces the effectiveness of the snail's venom and results in the capture of less prey (food). Therefore, such a mutation will be selected against and eventually be eliminated from the population. In contrast, a mutation that enhances the effectiveness or function of a toxin will increase the fitness of the organism (increase prey capture) and will be positively selected.

Because deleterious mutations are eventually eliminated and advantageous mutations are selected for, the invention utilizes allowed substitutions as a source of variation having positive effects and likely retaining the function of the peptide (e.g., venom toxin). The novel approach of the invention allows the number of peptides required to be dramatically reduced and increases the percentage of functional peptides.

In the hypothetical example, where four (4) positions are non-conserved, the combinatorial approach required 160,000 sequences. Using the method of the invention and assuming that three (3) different amino acids (allowed substitutions) are found in each of the four non-conserved positions, only (3×3×3×3=81) 81 sequences are required. Thus, the invention provides a dramatic reduction in the number of sequences required (160,000 versus 81). If the hypothetical is altered to assume five different amino acids at each non-conserved position, only (5×5×5×5=625) 625 sequences are required. Additionally, these sequences are highly likely to retain function. As will be recognized, the practical considerations of generating 81 or even 625 sequences, as opposed to 160,000, provides the artisan with a tremendous advantage.

Thus, the invention provides the practical ability to synthesize the required peptides and increases the concentration of any one sequence in the set. The invention also includes a set (library) of sequences having new sequences likely to confer an activity not present in the original representatives.

In contrast to the combinatorial approach, a individual member approach tests the function of individual members one at a time. The individual member approach reduces the number of sequences to one and makes the synthesis of the sequence readily obtainable. However, the individual approach is necessarily limited to ascertaining the function and properties of that one member. The approach provides no information regarding the function of sequences other that the tested sequence. In addition, the individual approach may not identify a function where the assay and the individual member selected are incompatible. Furthermore, the individual member approach is extremely labor and time intensive where multiple sequences must be assayed for different molecular targets. Moreover, the individual member approach cannot address combinations of allowed substitutions not present in an identified sequence. Thus, the invention provides significant advantages over the individual member approach and performs a different function.

In one embodiment, the sequences of the invention, for example, those sequences derived from Conus, are useful in the treatment of disease and adverse medical conditions. Numerous diseases have been proposed to be treated with conopeptides, including neurological disorders, such as epilepsy, multiple sclerosis, Parkinson's disease, Huntington's disease, schizophrenia, and other conditions, such as pain, anxiety, depression and sleep disorders. Neuronal receptors, such as the NMDA receptor, have been reported to play significant roles in these diseases. Furthermore, receptors, such as the NMDA receptor, exist in multiple subtypes. These subtypes frequently bind to different molecules (antagonists or agonists) and provide different responses. Because receptors exist as different subtypes, the treatment of many diseases is best accomplished by the production of highly selective treatments that effect specific receptor subtypes.

A major problem in medicine results from side effects that drugs very often exhibit, some of which are caused by the drug binding not only to the particular receptor subtype that renders therapeutic value, but also to closely related, therapeutically irrelevant receptor subtypes which can often cause undesirable physiological effects. In contrast to most drugs, conopeptides generally discriminate among closely related receptor subtypes.

Thus, peptides of the invention, for example, conopeptides, may bind to a specific receptor or receptor subtype and can be used to target specific receptors. In addition, the peptides may be used in assays for this receptor. Further, the peptides of the invention may be assayed to identify specific binding to a single receptor subtype and for reduced or no binding to different receptor subypes. Thus, the peptides of the invention relating to conopeptides and venom peptides in general have a particularly useful characteristic of high affinity for a particular macromolecular receptor, accompanied by a narrow receptor-subtype specificity. The pharmacological specificity of the conotoxins makes them attractive for drug development for a variety of therapeutic applications, including neurological and cardiovascular disorders.

In addition, the peptides of the invention provide combinations of allowed substitutions that can have specificity to new receptor subtypes, different binding affinities and have different properties (e.g., different off rates).

In another embodiment, the invention provides nucleic acid sequences which encode a set of polypeptides. The skilled artisan may convert between nucleic acid and amino acid sequences, for example, a known nucleic acid sequence may be used to determine the presumed gene product or a known polypeptide sequence may be used to determine a nucleic acid that will encode the polypeptide. Thus, the nucleic acid sequences of the invention may be analyzed relative to encoded polypeptides and/or designed to reflect the degeneracy of the genetic code.

In another embodiment, the invention provides nucleic acid sequences having a function independent of encoding a polypeptide. For example, the nucleic acid sequences may encode a telomerase RNA molecule. In this example, telomerase RNA sequences (possibly including pseudogenes) would be aligned and the observed members at each position of the alignment identified. A set of nucleic acid sequence combinations is generated, wherein the set is the union of observed members at each position. The set of nucleic acid sequence combinations is then assayed for a desired function. For example, the set of telomerase RNA sequences may be assayed for decreased telomerase activity, thereby identifying and generating a potential anti-cancer product.

Thus, the invention provides nucleic acid sequences which encode proteins having a desired property and nucleic acids which themselves provide a desired property.

Terms and Definitions

As used herein, the terms “conotoxin,” “conotoxin polypeptide,” and conopeptide includes conantokin peptides, conantokin peptide derivatives, conotoxin peptides (including, contryphans, bromocontryphans, contulakins, conophysins, conopressins and conorfamides) and conotoxin peptide derivatives. Conotoxins are typically derived from the venom of Conus snails, and may include one or more amino acid substitutions, deletions and/or additons. These peptides may be referred to in the literature as conotoxins, conantokins or conopeptides. The conotoxin may be produced by methods, such as, in vitro translation, in vitro transcription and translation, recombinant expression systems, and chemical synthesis.

As used herein “substantially pure” means a preparation which is at least 60% by weight (dry weight) the compound or set of compounds of interest, for example, a nucleic acid, polypeptide or set of polypeptides or nucleic acids. Preferably, the preparation is at least 75%, more preferably at least 90%, and most preferably at least 99% by weight the compound of interest. Purity can be measured by any appropriate method (e.g., column chromatography, polyacrylamide gel electrophoresis, or HPLC analysis).

As used herein an “isolated nucleic acid” means a nucleic acid that is not immediately contiguous with both of the coding sequences with which it is immediately contiguous (one on the 5′ end and one on the 3′ end) in the naturally-occurring genome of the organism from which it is derived. For example, the term includes a recombinant nucleic acid which is incorporated into a vector; into an autonomously replicating plasmid or virus; or into the genomic DNA of a prokaryote or eukaryote; or a recombinant nucleic acid which exists as a separate molecule (for example, a cDNA or a genomic DNA fragment produced by PCR or restriction endonuclease treatment) independent of other sequences. It also includes a recombinant nucleic acid which is part of a hybrid gene encoding additional polypeptide sequence. The nucleic acid sequences may be RNA or DNA.

As used herein “positioned for expression” means that the nucleic acid molecule is operably linked to a sequence which directs transcription and, where appropriate, translation of the nucleic acid molecule.

“Probes” are molecules capable of interacting with a target nucleic acid, typically in a sequence specific manner, for example through hybridization. The hybridization of nucleic acids is well understood in the art. Typically a probe can be made from any combination of nucleotides or nucleotide derivatives or analogs available in the art.

As used herein “specifically binds” means a molecule which binds to a target, but which does not substantially recognize and bind other molecules in a sample (for example, a biological sample).

As used herein “peptide,” “polypeptide” and “protein” (which, at times may be used interchangeably herein) include polymers of two or more amino acids (whether or not naturally occurring) linked via a peptide bond. No distinction, based on length, is intended between a peptide, a polypeptide or a protein. In addition, proteins comprising multiple polypeptide subunits (e.g., DNA polymerase III, RNA polymerase II) or other components (e.g., an RNA molecule, as occurs in telomerase) are included within the meaning of “protein” as used herein. Similarly, fragments of a protein and polypeptide are also within the scope of the invention and may be referred to herein as “peptide,” “polypeptide” or “protein.”

A particular amino acid sequence of a given protein (i.e., the polypeptide's “primary structure,” when written from the amino-terminus to carboxy-terminus) may be determined by the nucleotide sequence of the coding portion of a mRNA, which is in turn specified by genetic information, typically genomic DNA (including organelle DNA, e.g., mitochondrial or chloroplast DNA). Alternatively, a nucleic acid (DNA or RNA) may be derived from the amino acid sequence of a peptide.

As used herein “receptor” means a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors include, but are not limited to, antibodies (e.g., monoclonal antibodies, polyclonal antibodies and antisera reactive with specific antigenic determinants (such as on viruses, cells or other materials)), cell membrane receptors (for example, voltage-gated or ligand-gated receptors, such as nicotinic receptors, gamma-aminobutyric acid (GABA) receptors, glycine receptors, glutamate receptors, serotonin receptors, α-bungarotoxin receptors, muscarinic receptors, N-methyl-D-aspartate (NMDA) receptors, nicotinic acetylcholine (nACh) receptors), voltage-gated ion channels, sodium channels, calcium channels, potassium channels and the like.

Receptors are frequently assembled from multiple subunits, for example, the nAChRs are assembled from five subunits arranged around a central cation-conducting pore. In muscle only one subtype has been identified, which is composed of two α, one β, one δ and one γ subunit. In contrast, eight neuronal nAChRs α subunits (α₂-α₇ and α₉-α₁₀) and three β subunits (β₂-β₄) have been identified in mammalian systems. These can be assembled as homomers (for example, α₇ or α₉), binary heteromers (for example, α₃β₂, α₂β₄ or α₄β₂) or complex heteromers (for example, α₃α₅β₂ or α₃β₃β₄). Receptor subtypes may be differentially distributed throughout the central and peripheral nervous system. Different conopeptides are known to selectively target nAChRs. The conopeptides identified to date which specifically bind nAChRs are antagonists that fall into two classes: those that act at the ACh site and those that bind noncompetitively as pore blockes. However, ligands selected for binding to a receptor, for example, a conopeptide, may act as either an antagonist, blocking an action, or an agonist, eliciting an action.

As used herein, a “Ligand Receptor Pair” or “Ligand binding pair” is formed when two macromolecules have combined through molecular recognition to form a complex. A ligand binding pair member is one of the two macromolecules forming the ligand binding pair.

Sequence identity is typically measured using sequence analysis software (e.g., Sequence Analysis Software Package of the Genetics Computer Group, University of Wisconsin Biotechnology Center, 1710 University Avenue, Madison, Wis. 53705, or BLAST software available from the National Library of Medicine). Examples of useful software include, but are not limited to, the GCG (Genetics Computer Group, Madison Wis.) program package (Devereux, J., et al., 1984), BLASTP, BLASTN, FASTA (Altschul et al., 1990); Altschul et al., 1997), PILE-UP and PRETTYBOX. The well-known Smith-Waterman algorithm may also be used to determine identity. Such software matches similar sequences by assigning degrees of homology to various substitutions, deletions, additions, and other modifications. While there exist a number of methods to measure identity between two polynucleotide or polypeptide sequences, the term “identity” is well known to skilled artisans. Preferred methods to determine identity are designed to give the largest match between the two sequences tested. Such methods are codified in computer programs. Conservative substitutions typically include substitutions within the following representative groups: glycine, alanine; valine, isoleucine, leucine; aspartic acid, glutamic acid, asparagine, glutamine; serine, threonine; lysine, arginine; and phenylalanine, tyrosine. It is understood that other groups known in the art may also constitute conservative substitutions.

The terms “homologous” or “homologue” or “ortholog” or “paralog” refer to related sequences that share a common ancestor or arise from gene duplication and are determined based on degree of sequence identity. Alternatively, a related sequence may be a sequence having homology, which has arisen by convergent evolution. These terms describe the relationship between a gene found in one species, subspecies, variety, cultivar or strain and the corresponding or equivalent gene in another species, subspecies, variety, cultivar or strain or, in the case of paralogous genes, two related sequences within a species, subspecies, variety, cultivar or strain. For purposes of describing the invention the term “homologous” includes orthologs and paralogs. “Homologous sequences” are thought, believed, or known to be functionally related. A functional relationship may be indicated in a number of ways, including, but not limited to: (a) the degree of sequence identity; and/or (b) the same or similar biological function. Preferably, both (a) and (b) are indicated. The degree of sequence identity may vary, but is preferably at least 50% over the region defining the relationship (when using standard sequence alignment programs known in the art), preferably between about 60% to about 99%, more preferably between about 75% to about 99%, even more preferably between about 85% to about 99%. Homology can be determined using software programs readily available in the art, such as those discussed in Current Protocols in Molecular Biology (F. M. Ausubel et al., eds., 1987). Preferred alignment programs are MacVector (Oxford Molecular Ltd, Oxford, U.K.) and ALIGN Plus (Scientific and Educational Software, Pennsylvania). Another alignment program is Sequencher (Gene Codes, Ann Arbor, Mich.), using default parameters.

Phylogenetically related sequences are sequences, either nucleic acid or amino acid, which are homologous sequences. Phylogenetically related sequences may be defined based on a specific domain (e.g., kinase domains), signal sequences, structural motifs (e.g., the cysteine motifs of conopeptides), and/or homology in untranslated regions such as the 5′ or 3′ UTR. Phylogenetically related sequences may be related by any evolutionary distance, preferably the sequences are closely related and more prefereably are from the same genus. Preferably, phylogenetically related sequences are selected from a clade. Evolutionary distance or phylogenetic distance can be calculated using computer algorithms such as, PHYLIP (Felsenstein, J. 1989), PAUP (Swofford, D. L., 1993; Swofford, D. L., 1998), MEGA (Kumar et al., 1993), and the like. See WEN-HSIUNG LI, 1997.

For example, conopeptides may be aligned based on sequence conservation in the signal sequence, the 3′UTR, the cysteine architecture and optionally the pro-domain. Alignment of conopeptides using the nucleic acid sequence coding for the mature toxin, using information generated by silent base changes, may also be used to generate an alignment. Alternatively, an alignment may be generated from the amino acid sequence of the peptide. For example, alignment of the amino acid sequence of mature toxins may be used to generate an alignment. Thus, the invention utilizes the 3′ UTR and signal sequence to generated phylogenetic relationships between conopeptides, where the cysteine scaffold serves to verify the alignment. Thus, one representative embodiment of the invention is the generation of phylogenetic relations between conopeptides.

The term hybridization typically means a sequence driven interaction between at least two nucleic acid molecules in a nucleotide specific manner, such as a primer or a probe and a gene. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

A characteristic structural feature of conotoxins is a large number of posttranslational modifications, in particular disulfide bridges. The primary function of disulfide bonds appears to be stabilization of the structure. Conotoxins are grouped into families, based upon the number and arrangement of disulfides bonds. For example, two-disulfide containing α-conotoxins contain the cysteine pattern, CC—C—C, with disulfides between 1^{st} and 3^{rd}, 2^{nd} and 4^{th} cysteines. Three-disulfide containing ω- and δ-conotoxins share the native cysteine pattern, C—C—CC—C—C, whereas μ-conotoxins share the common cysteine pattern, CC—C—C—CC. For native ω-, δ-conotoxins, the 1^{st} & 4^{th}, 2^{nd} & 5^{th} and 3^{rd} & 6^{th} cysteines are connected, for native μ-conotoxins the 1^{st} & 4^{th}, 2^{nd} & 5^{th} and 3^{rd} & 6^{th} cysteines are connected by disulfide bonds. The correct pairing of disulfides in the native conotoxins has been viewed as a prerequisite for maintaining their biological activity. However, non-native disulfide bonds The disulfide bridges are formed in a process of oxidative pairing of the cysteine residues. The conopeptides may be grouped according to the following superfamily and family structure:

| Superfamily | Family | Target | Cysteine Structure |
|---|---|---|---|
| O | δ | Na Channels | C—C—CC—C—C |
| | μO | Na Channels | C—C—CC—C—C |
| | κ | K Channels | C—C—CC—C—C |
| | ω | Ca Channels | C—C—CC—C—C |
| | γ | Pacemaker Channels | C—C—CC—C—C |
| M | μ | Na Channels | CC—C—C—CC |
| | Ψ | nACh Receptors | CC—C—C—CC |
| A | α | nACh Receptors | CC—C—C |
| | P | α₁-adrenergic Receptors | CC—C—C |
| | αA | nACh Receptors | CC—C—C—C—C |
| | κA | K Channels | CC—C—C—C—C |
| S | σ | 5HT₃ Receptors | C—C—C—C—C—C—C—C—C—C |
| I | ι1 | Unknown | C—C—CC—CC—C—C |
| | I2 | Unknown | C—C—CC—CC—C—C |
| P | ? | Unknown | C—C—C—C—C—C |
| T | τ | Unknown | CC—CC |
| | χ | Noradrenaline transporter | CC—C—C |
| Z | ? | Unknown | C—C—C—C |

Homologous sequences may have different lengths, which may be viewed as an insertion or deletion in one or the other sequence. Since an insertion in one sequence can always be seen as a deletion in the other, the term “indel” is frequently used to describe this situation. The result of an indel is that a position or a stretch of positions may be paired up with dashes (the gap-character) in the other sequence to signify such an insertion or deletion. Indels are assigned “gap penalties,” which are known in the art and incorporated into computer programs used in determining homology.

Phylogenetically related sequences may be subdivided based on any appropriate criteria, for example, phylogenetic distance, function, motif organization, or the like. Selection of the most appropriate phylogenetically related sequences is known by a person of skill in the art and determined by such persons.

A person of skill in the art may select or set the criteria for grouping related sequences as appropriate for the situation. When large numbers of individual sequences have been identified it is possible for the skilled artisan to identify robust phylogenetic groupings for related sequences. Particularly, for related sequences having pronounced hypervariable regions, such as conopeptides. For example, sequences within a superfamily may be further divided into families and/or subfamilies and even further divided into evolutionarily closer clades. Sequences having a robust phylogenetic relationship, for example, as expressed by relatively short evolutionary distances within the group, will likely perform the same function or affect the same target (for example, the same receptor subtype). Therefore, restricting the phylogenetic group or a clade to more closely related sequences serves to focus on a particular function or target and identifies allowed substitutions which likely retain activity against the target. The ability of a skilled artisan to set the criteria used to establish a group of phylogenetically related sequences allows the methods and compositions disclosed herein to be applicable to a vast range of sequences.

The term “nucleotide change” refers to one or more nucleotide substitution, deletion, and/or insertion, as is well understood in the art.

The proteins of the invention may be co-translationally, post-translationally or spontaneously modified. The peptides of the invention may be synthesized using modified amino acids or be modified subsequent to synthesis. For example, by C-terminal amidation, hydroxylation of proline, γ-carboxylation of glutamate, L- to D-epimerisation of naturally occurring amino acids, regioisomeric type bromination at a indolic C-6 position of L-tryptophan, acetylation, famesylation, glycosylation, myristylation, methylation, prenylation, phosphorylation, palmintolation, sulfation, ubiquitination and the like. See, Wold, F., 1981. In addition, the proteins of the invention may be synthesized using modified or non-natural amino acids and derivatives. For example, a large number of non-natural or unusual amino acids are available from Chem-Impex International, Inc. (catalog available online at http://www.chemimpex.com), including: 2-Acetylamino-6-N-Boc-amino-4-hexynoic acid; Aminobenzoic acid (Abz); 4-Amino-1-benzoyl-pyrrolidine-2-carboxylic acid (AZPC); 3-Amino-3-(4-bromophenyl)-propionic acid; Aminobutyric acid (Abu); e-Aminocaproic acid (e-Acp) (6-Aminohexanoic acid); 6-Amino-2-carboxymethyl-3,8-diazabicyclo-(4,3,0)-nonane-1,4-dione (Acdn); Aminocyclohexane carboxylic acid; 4-Amino-2-cyclopentene-1-carboxylic acid; 1-Aminocyclopropane-1-carboxylic acid (Acpc); 10-Aminodecanoic acid; 2-Aminohexanoic acid; 4-Amino-3-hydroxy-6-methylthio-hexanoic acid (AHTHxA); δ-Aminolevulinic acid; Aminomethyl benzoic acid (Amb); 4-Aminomethyl-phenylacetic acid; 3-Amino-3-(1-naphthyl)-propionic acid; 8-Aminooctanoic acid; 3-Amino-2-oxo-1-azepine-acetic acid; Aminooxyacetic acid (Aoa)); 4-Aminopiperidine-4-carboxylic acid (Pip)); 3-Aminopropyl bromide; 3-Amino-3-(3-pyridyl)-propionic acid; 4-Amino-pyrrolidine-2-carboxylic acid (ABPC); a-Aminosuberic acid (Asu) (2-Aminooctanedioic acid); 2-Aminotetraline-2-carboxylic acid (Atc); 2-Amino-2-thiazoline-4-carboxylic acid; 11-Aminoundecanoic acid; Citrulline (Cit); 1,2-Diaminoethane; 1,3-Diaminopropane; 1,3-Dihydro-2H-isoindole carboxylic acid (Disc); Homocitrulline (HoCit) (N-e-Carbamyl-lysine); Hydroxylamine; 6-Hydroxynorleucine; 4-Hydroxyproline (Hyp); 3-Phenylazetidine-2-carboxylic acid (racemic); Phenylglycine (Phg); Propanolol; Pyroglutamic acid (Pyr); Sarcosine (Sar) (N-Methylglycine); Selenoethionine; 1,2,3,4-Tetrahydroisoquinoline-1-carboxylic acid (Tiq); 1,2,3,4-Tetrahydronorharman-3-carboxylic acid (Tpi); Thiazolidine-4-carboxylic acid (Thz); Thiazolidin-2-one-4-carboxylic acid; Thyronine; 1,2,3,4-Tetrahydroisoquinoline-3-carboxylic acid (Tic); and Tranexamic acid.

For the purposes of this invention, the source of the polynucleotide from an organism or its ancestor can be any suitable source, for example, genomic sequences or cDNA sequences. Preferably, cDNA sequences are compared. For the purposes of this invention, the source of the polypeptide from the organism or its ancestor can be any suitable source, for example defined tissues or cells, intracellular or exctracellular material, or recombinant expression systems. Polypeptide sequences may be determined by direct sequencing of the polypeptide, for example by Edmond degradation or Mass spectrometry (MS), or by deriving the sequence from the nucleic acid encoding the polypeptide. Nucleic acid or polypeptide sequences can be obtained from available private, public and/or commercial databases. These databases serve as repositories of the molecular sequence data generated by ongoing research efforts.

Nucleic acid or polypeptide sequences may be obtained from, for example, sequencing of cDNA reverse transcribed from mRNA expressed in cells, or after PCR amplification, according to methods well known in the art (using, for example, GeneAmp PCR System 9700 thermocyclers (Applied Biosystems, Inc.)). Alternatively, genomic sequences may be used for sequence comparison.

In some embodiments, the cDNA is prepared from mRNA obtained from a specific tissue, a tissue at a determined developmental stage or a tissue obtained after the organism has been subjected to certain conditions. cDNA libraries used for the sequence comparison of the present invention can be constructed using conventional cDNA library construction techniques that are explained fully in the literature of the art. Total mRNAs may be used as templates to reverse-transcribe cDNAs. Transcribed cDNAs may be subcloned into appropriate vectors to establish a cDNA library. The established cDNA library can be maximized for full-length cDNA contents, although less than full-length cDNAs may be used. Furthermore, the sequence frequency can be normalized according to, for example, Bonaldo et al., 1996. cDNA clones randomly selected from the constructed cDNA library can be sequenced using standard automated sequencing techniques. Preferably, full-length cDNA clones are used for sequencing.

In one embodiment of the invention, cDNA clones to be sequenced can be pre-selected according to their expression specificity. In order to select cDNAs corresponding to active genes that are specifically expressed, the cDNAs can be subject to subtraction hybridization using mRNAs obtained from other organs, tissues or cells of the same animal. Under certain hybridization conditions, with appropriate stringency and concentration, those cDNAs that hybridize with non-tissue specific mRNAs, and thus likely represent “housekeeping” genes, will be excluded from the cDNA pool. Accordingly, remaining cDNAs to be sequenced are more likely to be associated with tissue-specific functions. For the purpose of subtraction hybridization, non-tissue-specific mRNAs can be obtained from one organ, or preferably from a combination of different organs and cells. The amount of non-tissue-specific mRNAs are maximized to saturate the tissue-specific cDNAs.

In another embodiment of the invention, sequences can be pre-selected by using PCR primers which are specific to the desired class of sequences. For PCR, primers may be made from one or more organism's sequences using standard methods in the art, including publicly available primer design programs such as PRIMER.RTM. (Whitehead Institute). The amplified sequence may then be sequenced using standard methods and equipment in the art, such as automated sequencers (Applied Biosystems, Inc.).

Alternatively, information from online databases can be used to select or give priority to cDNAs that are more likely to be associated with specific functions. For example, the cDNA candidates for sequencing can be selected by PCR using primers designed from representative candidate cDNA sequences. Representative candidate cDNA sequences are, for example, those that are only found in a specific tissue, such as venum duct, or that correspond to genes likely to be important in the specific function. Such tissue-specific cDNA sequences may be obtained by searching online sequence databases in which information with respect to the expression profile and/or biological activity for cDNA sequences may be specified.

The peptides of the invention may be synthesized by a suitable method, such as by exclusively solid-phase techniques, by partial solid-phase techniques, by fragment condensation or by classical solution couplings. The employment of recombinant DNA techniques may be used to prepare these peptides, particularly longer ones.

In conventional solution phase peptide synthesis, the peptide chain can be prepared by a series of coupling reactions in which the constituent amino acids are added to the growing peptide chain in the desired sequence. The use of various N-protecting groups, various coupling reagents (e.g., dicyclohexylcarbodiimide or carbonyldimidazole), various active esters (e.g., esters of N-hydroxyphthalimide or N-hydroxy-succinimide), and the various cleavage reagents, to carry out reaction in solution, with subsequent isolation and purification of intermediates, is well known classical peptide methodology. Classical solution synthesis is described in detail in the treatise GEORG THIEME. VERLAG, STUTTGART, W. GER, 1974. Techniques of exclusively solid-phase synthesis are set forth in the textbook SOLID-PHASE PEPTIDE SYNTHESIS, and are exemplified by the disclosure of U.S. Pat. No. 4,105,603. The fragment condensation method of synthesis is exemplified in U.S. Pat. No. 3,972,859. Other available syntheses are exemplified by U.S. Pat. No. 3,842,067 and U.S. Pat. No. 3,862,925. The above references are herein incorporated by reference.

Common to such chemical syntheses is the protection of the labile side chain groups of the various amino acid moieties with suitable protecting groups which will prevent a chemical reaction from occurring at that site until the group is ultimately removed. Usually also common is the protection of an alpha-amino group on an amino acid or a fragment while that entity reacts at the carboxyl group, followed by the selective removal of the alpha-amino protecting group to allow subsequent reaction to take place at that location. Accordingly, it is common that, as a step in such a synthesis, an intermediate compound is produced which includes each of the amino acid residues located in its desired sequence in the peptide chain with appropriate side-chain protecting groups linked to various of the residues having labile side chains.

As far as the selection of a side chain amino protecting group is concerned, generally one is chosen which is not removed during deprotection of the α-amino groups during the synthesis. However, for some amino acids (e.g., His) protection is not generally necessary. In selecting a particular side chain protecting group to be used in the synthesis of the peptides, the following general rules are followed: (a) the protecting group preferably retains its protecting properties and is not split off under coupling conditions, (b) the protecting group should be stable under the reaction conditions selected for removing the α-amino protecting group at each step of the synthesis, and (c) the side chain protecting group must be removable, upon the completion of the synthesis containing the desired amino acid sequence, under reaction conditions that will not undesirably alter the peptide chain.

The C-terminal amino acid, protected by Boc and by a side-chain protecting group, if appropriate, can be first coupled to a chloromethylated resin according to procedures known in the art (See, Hlavacek and Ragnarsson, 2001). For example, using KF in DMF at about 60° C. for 24 hours with stirring, when a peptide having free acid at the C-terminus is to be synthesized. Following the coupling of the BOC-protected amino acid to the resin support, the α-amino protecting group is removed, as by using trifluoroacetic acid (TFA) in methylene chloride or TFA alone. The deprotection is carried out at a temperature between about 0° C. and room temperature. Other standard cleaving reagents, such as HCl in dioxane, and conditions for removal of specific α-amino protecting groups may be used as described in SCHRODER & LUBKE, 1965.

Cyclization of the linear peptide is preferably effected, as opposed to cyclizing the peptide while a part of the peptidoresin, to create bonds between Cys residues. To effect such a disulfide cyclizing linkage, the fully protected peptide can be cleaved from a hydroxymethylated resin or a chloromethylated resin support by ammonolysis, as is well known in the art, to yield the fully protected amide intermediate, which is thereafter suitably cyclized and deprotected. Alternatively, deprotection as well as cleavage of the peptide from the above resins or a benzhydrylamine (BHA) resin or a methyl-benzhydrylamine (MBHA) can take place at 0° C. with hydrofluoric acid (HF), followed by air-oxidation under high dilution conditions.

The disulfide bonds present in conopeptides and other proteins may also be generated by air-oxidation of the linear peptides for prolonged periods under cold room temperatures. Therefore, a method for making disulfide containing peptides includes oxidizing the linear peptide and then fractionating the resulting product, using reverse-phase high performance liquid chromatography (HPLC) or the like, to separate peptides having different disulfide linkage configurations. By comparing these fractions with the elution of the native material or by using a simple assay, the particular fraction having the correct linkage for maximum biological potency may be determined.

The preparation of a Conus textile venom duct cDNA library has been described (Sasaki et al., 1999). For example, venom duct mRNA may be prepared from specimens from any species (e.g., Conus arenatus, Conus pennaceus, Conus tessulatus, and Conus ventricosus) (Conticello et al. 2001). cDNAs may be prepared by oligo dT (with or without a restriction site) priming and/or ligated to adaptors, and cloned into an appropriate vector. Clones from the library may then be sequenced, for example, by the dye terminator method on ABI 373 or ABI 377 automated sequencers.

Sequences may be edited to discard vector and adaptor regions using computer programs, such as, Sequencher 3.0 (GeneCodes Corp., Ann Arbor, Mich.). Contigs (the assembly of individual sequences into a contiguous sequence) may be assembled manually or automatically, with or without subsequent manual edition. Individual transcripts are typically aligned using computer programs, such as, CLUSTAL X (Thompson et al. 1997), and the alignments may be refined manually. Phylogenetic trees may be constructed using the neighbor-joining method (Saitou and Nei, 1987) and visualized with computer programs, such as, TreeView (Page, 1996). Synonymous versus nonsynonymous substitution rates may be analyzed using MEGA (Kumar, et al. 1993). To estimate the significance of differences in substitution rates in the different regions, both within and between species and gene families, a one-tailed t-test with infinite degrees of freedom may be used. Tip tests (Templeton, 1996) are performed on the basis of alignments specific to the analyzed region (for example, signal+propeptide, mature domain) in order to reduce the complexity of the cladogram (for example, clades in signal+propeptide-based trees are different from the clades in mature-peptide based trees). A Fisher 2×2 contingency test, for example, as suggested by Castelloe and Templeton, 1994, is performed on silent versus replacement substitutions in external and internal branches of the gene tree.

RT-PCR is performed using primers that anneal to conserved elements in the 5′ and 3′ untranslated regions (UTRs) of each conopeptide family. Conditions for RT-PCR are determined by a person of skill in the art and may include: 50° C. for 40 min and 94° C. for 2 min, followed by 25 amplification cycles of 94° C. for 30 s, 55-60° C. for 30 s, and 68° C. for 1 min. The resulting PCR fragments may be ligated directly into a T-overhang vector, and clones from each reaction sequenced.

Where the molecular evolution or a determination of evolutionary distance between nucleic acids is sought, it is preferable to analyze the ratio of non-synonymous substitutions to synonymous substitutions. The ratio of non-synonymous substitutions to synonymous substitutions, the K_{A}/K_{S} ratio, may be carried out by the methods of Li et al., although other analysis programs that can detect positively selected genes between species can also be used. Li et al., 1985; Li, 1993; Messier and Stewart, 1997; Nei, 1987. The K_{A}/K_{S} method, which comprises a comparison of the rate of non-synonymous substitutions (K_{A}) per non-synonymous site with the rate of synonymous substitutions (K_{S}) per synonymous site between homologous protein-coding regions of genes in terms of a ratio, is used to identify sequence substitutions that may be driven by adaptive selections as opposed to neutral selections during evolution. A synonymous (“silent”) substitution is one that, owing to the degeneracy of the genetic code, makes no change to the amino acid sequence encoded. A non-synonymous substitution results in an amino acid replacement. The extent of each type of change can be estimated as K_{A} and K_{S}, respectively, the numbers of synonymous substitutions per synonymous site and non-synonymous substitutions per non-synonymous site. Calculations of K_{A}/K_{S} may be performed manually or by using software. An example of a suitable program is MEGA (Molecular Genetics Institute, Pennsylvania State University).

For the purpose of estimating K_{A} and K_{S}, either complete or partial protein-coding sequences are used to calculate total numbers of synonymous and non-synonymous substitutions, as well as non-synonymous and synonymous sites. The length of the polynucleotide sequence analyzed can be any appropriate length. Preferably, the entire coding sequence is compared, in order to determine any and all significant changes. Where appropriate and desirable, the comparison may be restricted to specific functional domains or the like. Publicly available computer programs, such as Li93 (Li(1993)) or INA, can be used to calculate the K_{A} and K_{S} values for all pairwise comparisons. This analysis can be further adapted to examine sequences in a “sliding window” fashion such that small numbers of important changes are not masked by the whole sequence. “Sliding window” refers to examination of consecutive, overlapping subsections of the gene (the subsections can be of any length).

K_{A}/K_{S} has been shown to be a reflection of the degree to which adaptive evolution has been at work in the sequence under study. The higher the K_{A}/K_{S} ratio, the more likely that a sequence has undergone adaptive evolution and that the non-synonymous substitutions are evolutionarily significant. See, for example, Messier and Stewart (1997).

Nucleic acid or polypeptide sequences are compared to identify homologous sequences. Any appropriate mechanism for completing this comparison is contemplated by this invention. Alignment may be performed manually or by software (examples of suitable alignment programs are known in the art).. Nucleic acid or polypeptide sequences may be selected for comparison via database searches (e.g., BLAST searches). The high scoring “hits,” i.e., sequences that show a significant similarity after BLAST analysis, will be retrieved and analyzed. Sequences showing a significant similarity can be those having at least about 60% to about 99% sequence identity over comparable regions. Preferably, sequences showing greater than about 80% identity are further analyzed. The homologous sequences identified via database searching can be aligned in their entirety using sequence alignment methods and programs that are known and available in the art, such as the commonly used simple alignment program CLUSTAL V by Higgins et al. 1992.

Alternatively, the sequencing and homology comparison of nucleic acid or polypeptide sequences may be performed simultaneously by sequencing chip technology. See, for example, U.S. Pat. No. 5,545,531.

The aligned nucleic acid or polypeptide sequences are analyzed to identify the nucleotide(s) or amino acid(s) observed at each position of the alignment. Again, any suitable method for achieving this analysis is contemplated by this invention. The detected sequence differences are generally checked for accuracy. Preferably, the initial checking comprises performing one or more of the following steps, any and all of which are known in the art: (a) finding the points where there are changes between two sequences; (b) checking the sequence fluorogram (chromatogram) or data source to determine if the bases or amino acids that appear unique to the sequence in question correspond to strong, clear signals specific for the called base or amino acid; and/or (c) checking additional sequences to see if there is more than one sequence that corresponds to a sequence change. Multiple sequence entries for the same gene or peptide that have the same nucleotide or amino acid at a position where there is a different nucleotide or amino acid in a reference sequence provides independent support that the sequence in question is accurate, and that the change is significant. Such changes may be examined using database information and the genetic code to determine whether these nucleotide sequence changes result in a change in the amino acid sequence of the encoded protein. As the definition of “nucleotide change” makes clear, the present invention encompasses at least one nucleotide change, a substitution, a deletion or an insertion, in a protein-coding polynucleotide sequence as compared to a corresponding sequence.

Newly identified significant changes within a nucleotide or polypeptide sequence, particularly in sequences subject to a high degree of selection pressure, may suggest a potential association with unique, enhanced or altered functional capabilities.

Nucleic acids encoding the peptides of the invention may be fused to reporter constructs, such as any of the Two-hybrid reporter systems or a display system, such as phage display. In addition, the nucleic acids may be fused to signal sequences or the like. The nucleic acids may also be inserted into a vector, including an expression vector, and, where appropriate, introduced into a prokaryotic or eukaryotic cell.

Functional nucleic acids which are included within the scope of the invention include, antisense molecules, aptamers, probes, ribozymes, triplex forming molecules, and external guide sequences. Aptamers are molecules that interact with a target molecule, preferably in a specific way (for a review see Gold et al., 1995, Annu. Rev. Biochem., 64, 763; and Szostak & Ellington, 1993, in The RNA World, ed. Gesteland and Atkins, pp 511, CSH Laboratory Press). Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a k_{d} less than 10⁻⁶. A representative example of how to make and use aptamers can be found in U.S. Pat. No. 6,458,559, hereby incorporated by reference.

Peptide libraries constructed by the method of the invention may be screened by iterative library analysis and resynthesis. The peptides of the library are pooled and screened for the desired activity, for example, for binding activity to a specific receptor. Once a desired activity is identified, the library is resynthesized as one or more pools of peptides having one variable amino acid held constant. The one or more pools of peptides are iteratively rescreened for the identified or desired activity. Screening or selecting may be performed by methods known in the art, such as phage-display, selectively infective phage, polysome technology to screen for binding, assay systems for enzymatic activity or protein stability. Polypeptides having the desired property can be identified by sequencing of the corresponding nucleic acid sequence or by amino acid sequencing.

The peptide libraries may also be screened for binding to one or more substance, with unbound peptide being removed (e.g., by washing the unbound peptides clear) and the bound peptides then eluted. The eluted peptides may then be rescreened using the same or more stringent conditions. This process may be repeated to achieve the desired binding specificity or activity. The peptide(s) eluted from the final round of selection may be sequenced, for example, using ionizing mass spectrometry or other methods known in the art.

A set of sequences may also be constructed using the methods disclosed herein, coupled with a location identification approach (e.g., an array or chip approach). According to this method, the peptides or nucleic acids of the library are synthesized and individual species are attached by known methods, such as covalently to microcarrier beads, as described in U.S. Pat. No. 5,143,854. Alternatively, the peptides or nucleic acids may be attached to multiwell supports or other known structures and physical supports. This method may be further modified by the method of Jayawickreme et al., 1994. The method of Jayawickreme et al. releases peptides bound to microcarrier beads using a gas-phase release procedure, thereby, facilitating the analysis of peptides wherein the peptide end, which was covalently attached to the microcarrier beads, is necessary for proper activity.

The peptides and nucleic acids of the invention may be linked to fluorescent compounds, such as fluorescein, Rhodamine, Texas Red, UV-light excitable dyes, quenching moieties and combinations thereof. Linking of fluorescent componds may be accomplished by methods known in the art, such as through use of amine-reactive probes. Alternatively, the peptides and nucleic acids may be labeled by other means known in the art, such as with radioactive isotopes, biotin, haptens, an antibody or fragment thereof, nonfluorescent dyes, enzymes (e.g., peroxidase and topoisomerase), peptides or chemicals. Additional amino acids and/or nucleotides may be added to the library at either end (the carboxy terminus, 5′ end, amino terminus or 3′ end) to facilitate attachment of a label. In addition the labels may be attached via a linker, such as carbon chains of between about 2 to 50 carbon atoms or the like.

The invention also provides for a kit, including one or more of the following: nucleic acid(s) of the invention; peptide(s) of the invention; recombinant vectors; suitable host cell(s), which may or may not contain nucleic acids of the invention, or express peptides of the invention; antibodies; receptors; computer programs; and methods for producing the peptides or nucleic acids of the invention.

The invention is further explained by the use of the following non-limiting illustrative examples.

### EXAMPLE I

### cDNA Library Construction

A cDNA library is constructed using appropriate cells, tissues or organisms. In addition, the cells or organisms may be temporally (e.g., in G2 of the cell cycle) or developmentally (e.g., insects in the third larval stage, organisms undergoing gastrulation, or the like) staged and harvested. A person of ordinary skill in the art may select the appropriate cells, tissue or organism to analyze according to the trait of interest. For example, venom producing cells are known to produce conopeptides useful for the treatment of disease. Thus, isolation of venom producing cells enriches for cells containing and expressing conopeptides.

In one embodiment, total RNA is extracted from Conus venom duct cells (RNeasy kit, Quiagen; RNAse-free Rapid Total RNA kit, 5 Prime—3 Prime, Inc.) and the integrity and purity of the RNA is determined according to conventional molecular cloning methods. Poly A+ RNA is isolated (Mini-Oligo(dT) Cellulose Spin Columns, 5 Prime—3 Prime, Inc.) and is used as a template for the reverse-transcription of cDNA with oligo (dT) as a primer. The synthesized cDNA is treated and modified for cloning using commercially available kits. Recombinants are then packaged and propagated in a host cell line. Portions of the packaging mixes are amplified and the remainder retained prior to amplification. The library can be normalized and the numbers of independent recombinants in the library determined.

### EXAMPLE II

Sequence Comparison

Suitable primers based on a candidate organism gene are prepared and used for PCR amplification of cDNA either from a cDNA library in a host cell line or from cDNA prepared directly from mRNA. Selected cDNA clones from the cDNA library are sequenced using an automated sequencer, such as an ABI 377. Primers, such as the M13 Universal and Reverse primers may be used to carry out the sequencing. Alternatively, the primers may be designed to anneal to one or more sites found in the cDNA. For inserts that are not completely sequenced by end sequencing, dye-labeled terminators or custom primers can be used to fill in remaining gaps. The sequences can also be examined by direct sequencing of the encoded protein.

DNA coding for conopeptides was isolated and cloned using conventional techniques and procedures known in the art, such as described in Olivera et al., 1996. For example, primers may be based on the DNA sequence of known conopeptides. DNA from single clones was amplified by conventional techniques using primers which correspond approximately to the M13 universal priming site and the M13 reverse universal priming site. Clones having a size of approximately 300-500 nucleotides were sequenced and screened for similarity to sequences of known conopeptides.

### EXAMPLE III

Table 1 shows the alignment of phylogenetically related conopeptides. As illustrated in Table 1, positions 3-5, 7-9, 13-15 and 17 are conserved, whereas positions 1-2, 6, 10-12, 16 and 18 exhibit different allowed substitutions (shown in bold).

The variable positions contain either 2 or 3 allowed substitutions. The sequences of the present example represent toxins used by snails of the predatory Conus genus to, among other things, immobilize their prey. Thus, the sequences are subject to strong natural selection. For example, the peptides must bind to the receptor of the various prey species with high affinity. Therefore, the allowed substitutions may reflect differences in the target receptors in the various prey. Alternatively, the allowed substitutions may reflect other selective advantages, such as a slow off rate or the like. By combining all allowed substitutions with all other allowed substitutions the invention provides for the identification of advantageous sequences not present in the original population without having to sample all possible combinations, including detrimental or non-allowed substitutions. Thus, using a library of all allowed amino acids provides a library with the greatest possible range of useful peptides without introducing random, and possibly deleterious, amino acids.

A representative alignment of sequences of conopeptides demonstrates the application of one embodiment of the invention.

TABLE 1

| | Position Number and |
|---|---|
| Sequence | Sequence |
| Name | 1---5----10---15-- |

| | | |
|---|---|---|
| Phylogenetically | Bt1.2 | GGCCSHPACAVNHPELCG |
| Related Sequences | | (SEQ ID NO: 1) |
| of α-conotoxins | Af1.1 | QECCSYPACNLDHPELCG |
| | | (SEQ ID NO: 2) |
| | Tx1.1 | PQCCSHPACNVDHPEICR |
| | | (SEQ ID NO: 3) |
| | R1.1B | PQCCSHPACNVDHPEICD |
| | | (SEQ ID NO: 4) |
| | Ae1.1 | PECCSHPACNVSHPEICR |
| | | (SEQ ID NO: 5) |
| | R1.1A | PECCSHPACNVDHPEICR |
| | | (SEQ ID NO: 6) |
| | | Variable positions and |
| | | variant amino acids |
| | | present at that position |
| | | (Number of represen- |
| | | tatives): |
| | | G(1) |
| | | Q(1) |
| | | P(4) |
| | | G(1) |
| | | E(3) |
| | | Q(2) |
| | | H(5) |
| | | Y(1) |
| | | A(1) |
| | | N(5) |
| | | V(5) |
| | | L(1) |
| | | N(1) |
| | | D(4) |
| | | S(1) |
| | | L(2) |
| | | I(4) |
| | | G(2) |
| | | R(3) |
| | | D(1) |

In this example, a library composed of the sequence Xaa₁ Xaa₂ Cys Cys Ser Xaa₃ Pro Ala Cys Xaa₄ Xaa₅ Xaa₆ His Pro Glu Xaa₇ Cys Xaa₈, (SEQ ID NO:7), wherein Xaa₁ is Gly, Qln or Pro; Xaa₂ is Gly, Glu or Qln; Xaa₃ is His or Tyr; Xaa₄ is Ala or Asn; Xaa₅ is Val or Leu; Xaa₆ is Asn, Asp or Ser; Xaa₇ is Leu or Ile; and Xaa₈ is Gly, Arg or Asp is prepared.

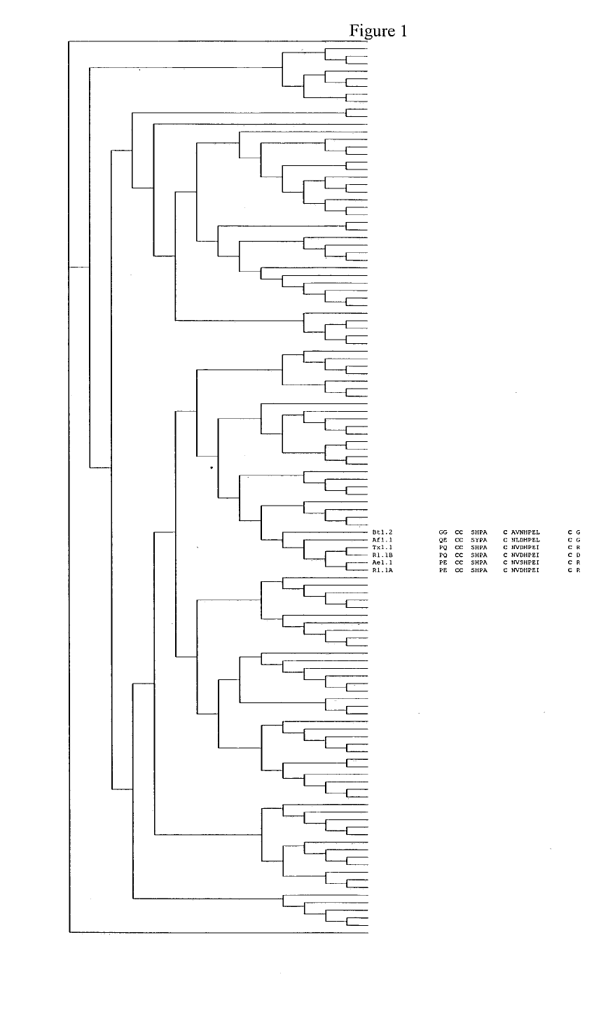

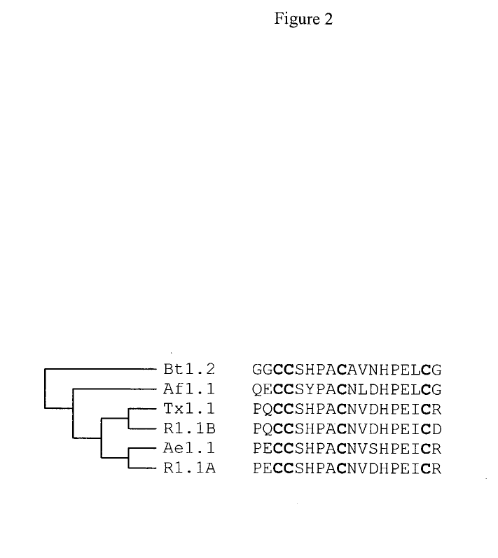

The phylogenetic relation of sequences shown in FIG. 1 was used to select phylogenetically related sequences. The selection of the phylogenetically related sequences may include any number of sequences desirable, based on the purpose of the desired library. In this example, the phylogenetically related sequences were selected based on their close phylogenetic relationship and as members of the a-conotoxin family, which interact with nicotinic acetylcholine receptors. Six initial sequences (FIG. 2) were selected. As shown in table 1, eight amino acid positions contained variable amino acids. Each of these variations has the potential to add specificity or increased activity. To test all of the possible combinations of allowed variation at each of the eight identified positions, a total of 1296 amino acid sequences are produced. In contrast, if the eight positions having allowed substitutions are randomized to place all amino acids in each position, a total of 2.5×10¹⁰ peptide sequences would have to be produced (assuming 20 natural amino acids). In the present example the sequences are chemically synthesized and folded by methods known in the art, including producing disulfide bonds between the appropriate cysteine residues.

TABLE 1.1

```
          α-conotoxin set of sequences (1296 sequences)
XXCCSXPACXXXHPEXCX SEQ ID NO: 7
GG H AVN L G
QE Y NLD I R
PQ S D
          3x3x2x2x2x3x2x3 = 1296 Sequences
```

Positions 3 to 5 are conserved, therefore, these three positions have one observed member and are fixed as Cys-Cys-Ser. Likewise, positions 7 to 9, 13 to 15, and 17 are conserved and non-variable or fixed amino acids. Positions 1-2, 6, 10-12, 16, and 18 exhibit different amino acids. For example position 6 exhibits two allowed amino acids, His (H) and Tyr (Y).

The invention includes a library representing a combination of all allowed substitutions. Position 1 is composed of one of the three allowed amino acids, which are combined with the three allowed amino acids of position 2, the two allowed amino acids observed at position 6, the two allowed amino acids observed at position 10, the two allowed amino acids observed at position 11, the three allowed amino acids observed at position 12, the two allowed amino acids observed at position 16, and the three allowed amino acids observed at position 18. Thus, the set of sequences representing the union of observed amino acids at each position results in 1296 (3×3×2×2×2×3×2×3) possible combinations of amino acids. Thus, to fully represent all observed amino acids at each position, a set of 1296 sequences is generated, having the sequence of SEQ ID NO:7.

When appropriate, for example, the library of the present Example, may be treated so as to form disulfide bonds (see U.S. application Ser. No. 10/377,332, filed Feb. 28, 2003, hereby incorporated by reference). The peptides may be treated with a protein disulfide isomerase to form the disulfide bonds. For example, bovine protein disulfide isomerase (PDI) is added to the set of peptides in 0.1M Tris/HCl, pH 7.5, containing 1 mM EDTA, 0.1 mM GSSG and 2 μM PDI, at 0° C. Alternatively, oxidative folding reactions of may be performed in 0.1M Tris/HCl, pH 8.7, containing 1 mM EDTA, 0.5 mM GSSG and 5 mM GSH, at 22° C. After an appropriate time the reaction is quenched by adding formic acid to the final concentration of 8%.

The person of skill in the art will recognize that synthesis of 1296 peptide sequences represents a significant improvement over synthesis of at least 2.5×10¹⁰ peptides as would be required by randomization. Thus, the invention reduces the number of peptides necessary to screen for function to a manageable number, without sacrificing the ability to identify advantageous properties or limiting the analysis to a single peptide, which may not function or may not have the desired properties as ascertained in the assay used. Furthermore, each peptide of the library will be present in a higher relative concentration, which facilitates the generation of the properly folded form of each representative sequence.

The library containing the 1296 representative sequences allows for the selection of evolutionarily non-represented species selected from a combination of all possible allowed amino acids for any position.

### EXAMPLE IV

### Study of Protein Function Using a Receptor Binding Assay

The function of a set of sequences (a library) may be determined by assaying the set (for example, the set of 1296 sequences disclosed in Example III). A receptor assay is created using an adaptation of the method described in McIntosh, 2000. Physiological, morphological and/or biochemical examination of the receptor will permit association of the library, and subsequently, each representative sequence, with a particular phenotype.

Conotoxin Library Binding: The conotoxin library of Example III is iodinated by the methods described in Cruz and Olivera, 1986. The binding protocol is a modification of that described in Hillyard et al., 1992. Crude brain membranes from Harlan Sprague-Dawley rats are prepared as described by Catterall, 1980, with modifications in buffer components as described in Cruz and Olivera, supra. The binding of ¹²⁵I-labeled conotoxin library sequences to rat brain membrane is measured in a 200-ml assay mix that contains approximately 10 mg of membrane protein, 100,000 cpm of carrier-free [¹²⁵I]conotoxin library peptides (approximately 150 pm), 0.2 mg/ml lysozyme, 0.32 M sucrose, 100 mM NaCl, and 5 mM HEPES/Tris (pH 7.4). Nonspecific binding is measured by preincubating the membrane preparation with 1 mM unlabeled conotoxin library for 30 min on ice before the addition of [¹²⁵I]conotoxin library. The library of Example III is assessed for activity by preincubating the library for 30 min on ice. The final assay mix is then incubated at room temperature for 30 min and diluted with 1.5 ml of wash buffer containing 160 mM NaCl, 1.5 mM CaCl₂, 2 mg/ml bovine serum albumin, 5 mM HEPES/Tris (pH 7.4). Membrane is collected on glass fiber filters (Whatman GF/C soaked in 0.1% polyethyleneamine), for example, using a Brandell apparatus model M-24, and washed with 1.5 ml of wash buffer four times. The amount of radioactivity in the filters is then measured.

The labeled library is screened against one or more targets. For example, the library is screened for α4β2 Nicotinic Acetylcholine Receptor (nAChR) binding. The procedure of Pabreza et al.,1991 is used. [³H]Cytisine (15-40 Ci/mmol) is obtained from a commercial supplier such as PerkinElmer Life Sciences. Rat forebrain membrane is incubated for 75 min at 4° C. in 50 mM Tris-HCl (pH 7.0 at room temperature) containing 120 mM NaCl, 5 mM KCl, 1 MM MgCl₂, and 2.5 mM CaCl₂. Nonspecific binding is defined with 10 mM nicotine.

The library is screened for Adrenergic α1 binding. Rat forebrain membranes are incubated with 0.3 nM [³H]prazosin (70-87 Ci/mmol). Reactions are carried out in 50 mM Tris-HCl (pH 7.7) at 25° C. for about 60 min. Prazosin (1.0 mM) is used to define nonspecific binding (19, 20).

The library is screened for Adrenergic a2 binding. Rat cortical membranes are incubated with 1.0 nM [³H]RX821002 (40-67 Ci/mmol). Reactions are carried out in 50 mM Tris-HCl (pH 7.4) at 25° C. for 75 min. RX821002 (0.1 mM) is used to define nonspecific binding (20, 21).

The library is screened for Adrenergic b1 binding. Rat cortical membranes are incubated with 0.2 nM (2)[¹²⁵I]iodopindolol (2200 Ci/mmol) and 120 nM ICI-118,551 (to block adrenergic b2 receptors). Reactions are carried out in 50 mM Tris-HCl (pH 7.5) containing 150 mM NaCl, 2.5 mM MgCl₂, and 0.5 mM ascorbate at 37° C. for about 60 min. Alprenolol HCl (10 mM) is used to define nonspecific binding (22, 23).

Each representative of the library has the potential to bind to a target. Thus, by conducting the described assays, one or more target for one or more members of the library is identified. Thus, the assay identifies ligand binding members. Because the library contains combinations of the allowed substitutions at two or more non-conserved positions in the phylogenetically related sequences the library is particularly suited to identify one or more functional target. Further, the library is particularly well suited to identify binding or increased binding to a subfamily of receptors.

The library member with the most desired binding property (e.g., highest binding affinity) is identified by binding assays performed under stringent conditions. The individual library member (ligand binding member) may be identified by iterative screening or by elution of the bound polypeptides, where possible, followed by peptide sequencing or mass spectrometry analysis.

### EXAMPLE V

### Phage Display Library Screening

One method of screening or selecting an amino acid or nucleic acid sequence is to introduce a nucleic acid or set of nucleic acids, which may include one or more degenerate sites, into a host cell. The host cell may then be screened or selected to identify one or more nucleic acid or amino acid sequences having desired properties. For example, amino acid sequences may be identified by phage display or use of an expression vector.

To display a library, such as a library encoding the polypeptides of Example III, on the surface of phage, synthetic genes encoding the representative polypeptides of the library are cloned into a filamentous phage vector, such as fdSN. The library polypeptides may be tethered at either their C termini or N termini to a carrier protein (e.g., the gene 3 protein of phage). Cultures containing fdSN and fusion phage constructs are tested to determine that the fusion of the library sequences to the carrier protein had no significant determinental effect on phage infectivity or packaging.

The amino acid sequence is backtranslated into a corresponding coding nucleic acid sequence individually or as a degenerate sequence. For example, the peptides of Example III may be generated using the following degenerate nucleic acid: 5′-NNA NNA UGC UGU UCC NAC CCC GCC UGC NNC NUC NNC CAC CCC GAG NUA UGC NNN-3′ (SEQ ID NO:8). A person of skill in the art will recognize that a number of other sequences may also be used, where the appropriate choice may be influenced by such factors as possible secondary structure in the RNA, melting temperature, codon preference in the host cell, and other factors known in the art.

Expression of the library on phage is determined by comparison of the Western blot band size of the carrier protein from helper phage, which contains the wild-type protein, and fd-library phage. To assess the functional activity of the library expressed on the phage surface, the binding activity of fd-library phage to a target such as the α4β2 Nicotinic Acetylcholine Receptor is tested. Because reducing the disulphide bonds of the library may abolish binding, the requirement for the disulphide scaffold for the functional activity of the library is tested. Phage incubated with 1% β-mercaptoethanol, or similar agents, to reduce the disulfide bonds of the fd-library, are tested for a reduction in binding affinity compared to non-treated phage.

When appropriate, the phage may be treated so as to increase disulfide bond formation. For example, phage may be treated with a protein disulfide isomerase or a protein disulfide isomerase may be expressed in the phage host cell.

The phage library is tested for receptor binding. Positive clones are selected and re-assayed for binding, with positive clones isolate at each successive round being re-screened. Individual colonies, for example, from round 4, are assayed for receptor binding. The DNA from these clones is then sequenced.

An identified clone is tested to determine whether the selected sequence retains function in the absence of phage. The identified sequence is isolated and expressed in Escherichia coli or synthesized. The protein sequence is isolated and retested for receptor binding.

The phage library may be constructed with a cleavage site between the carrier protein and the peptide. For example, one or more proteolytic cleavage site may be encoded by the nucleic acid (e.g., Endoproteinase Pro-Pro-Y-Pro, Factor X or Thrombin (available from Invitrogen). Alternatively, the peptides may be cleavable from the carrier protein by chemical cleavage.

### EXAMPLE VI

### Identification of Targets

The present invention is used to identify targets, including insect family specific targets. Insect-specific neurotoxins isolated from Australian funnel-web spiders have been reported. The w-ACTX-1 family of peptides (U.S. Pat. No. 5,959,182) each contain 36-37 residues with six strictly conserved cysteine residues that form three disulfide bonds. It is reported that a single species of spider may contain six or more variants of the toxin, with some variants differing by only a single conservative residue substitution. Wang, X.-H et al., 1999. The w-ACTX-1 family of toxins are reported to be lethal to a wide range of insects, including members from the orders Coleoptera, Orthoptera, Lepidoptera, and Diptera, but harmless when injected into newborn mice. Wang, X.-H et al. supra; Fletcher, J. I., et al., 1997; Tedford, H. W., et al., 2001. Injection of toxin into American cockroaches (Periplaneta americana) causes a loss of locomotion, high-frequency twitching of limbs with loss of righting reflexes, followed by paralysis and death. Fletcher, J. I., et al., supra. Direct application of toxin to the cockroach metathoracic ganglion abolishes hind-limb reflexes, whereas the forelimbs, which are not directly innervated by motor neurones of the metathoracic ganglion, are unaffected. Id. These peptides can therefore be classified as depressant neurotoxins. Electrophysiological studies revealed that the phylogenetic specificity of the toxins derives from their ability to block insect, but not vertebrate, voltage-gated calcium channels (VGCCs). Id. The channel subtype has been suggested to be an N- or L-like VGCC, but is not known.

### The Molecular Target of the w-ACTX-1 is Determined

The w-ACTX-1 family of peptides are aligned and the representative sequences of the library are determined, for example, as shown in Tables 2 and 2.1. A polypeptide library as previously described is constructed. The peptides of the library are then arrayed individually on a solid support. The array is then screened with receptor preparations, including N- and L-Voltage-gated calcium channels, and/or voltage-gated sodium channels and/or GABA receptors isolated from Heliothis armigera. Receptor binding is identified.

TABLE 2

| | | |
|---|---|---|
| Sequence Identity | Sequence | SEQ ID NOS: |

| | | |
|---|---|---|
| Accession Number | SAVCIPSGQP CPYSKYCCSG SCTYKTNENG NSVQRCD | SEQ ID NO: 9 |
| P81803 | | |
| Accession Number | SPTCIPSGQP CPYNENCCSQ SCTYKENENG NTVKRCD | SEQ ID NO: 10 |
| P81598 | | |
| Accession Number | SPTCIPSGQP CPYNENCCSK SCTYKENENG NTVQRCD | SEQ ID NO: 11 |
| P81597 | | |
| Accession Number | SSTCIPSGQP CPYNENCCSQ SCTFKENENG NTVKRCD | SEQ ID NO: 12 |
| P81596 | | |
| Accession Number | SSTCIPSGQP CPYNENCCSQ SCTYKENENG NTVKRCD | SEQ ID NO: 13 |
| P81595 | | |
| Accession Number | SPTCIPSGQP CPYNENCCSQ SCTFKENENG NTVKRCD | SEQ ID NO: 14 |
| P56207 | | |
| Observed Amino | SXXCIPSGGP CPYXXXCCSX SCTXKXNENG NXVXRCD | SEQ ID NO: 15 |
| Acids | | |

TABLE 2.1

| | |
|---|---|
| SXXCIPSGGP CPYXXXCCSX SCTXKXNENG NXVXRCD | SEQ ID NO: 15 |

| | | |
|---|---|---|
| Allowed Substitutions | | |
| at Each Position: | | |
| | AV SKY G Y T S Q | |
| | PT NEN Q F E T K | |
| | S K | |
| | 3x2 2x2x2x3x 2x2x 2x2 | = 2,304 Sequences |

The phylogenetically related sequences may be selected based on evolutionary distance. For example, Accession Number P81803 may be removed from the selected phylogenetically related sequences. In this case, Tables 3 and 3.1 illustrate the sequences which represent the combination of allowed amino acid substitutions. When Accession Number P81803 is removed from the phylogenetically related sequences, the set of sequence combinations having the union of observed amino acids at each position decreases from 2,304 to 24 sequences. Thus, the selection of the phylogenetically related sequences influences the complexity of the library.

TABLE 3

| | | |
|---|---|---|
| Sequence Identity | Sequence | SEQ ID NOS: |

| | | |
|---|---|---|
| Accession Number | SPTCIPSGQP CPYNENCCSQ SCTYKENENG NTVKRCD | SEQ ID NO: 10 |
| P81598 | | |
| Accession Number | SPTCIPSGQP CPYNENCCSK SCTYKENENG NTVQRCD | SEQ ID NO: 11 |
| P81597 | | |
| Accession Number | SSTCIPSGQP CPYNENCCSQ SCTFKENENG NTVKRCD | SEQ ID NO: 12 |
| P81596 | | |
| Accession Number | SSTCIPSGQP CPYNENCCSQ SCTYKENENG NTVKRCD | SEQ ID NO: 13 |
| P81595 | | |
| Accession Number | SPTCIPSGQP CPYNENCCSQ SCTFKENENG NTVKRCD | SEQ ID NO: 14 |
| P56207 | | |
| Conserved Amino | SXTCIPSGGP CPYNENCCSX SCTXKENENG NTVXRCD | SEQ ID NO: 16 |
| Acids | | |

TABLE 3.1

| | |
|---|---|
| SXTCIPSGGP CPYNENCCSX SCTXKENENG NTVXRCD | SEQ ID NO: 16 |

| | | |
|---|---|---|
| Allowed Substitutions | | |
| at Each Position: | | |
| | A G Y K | |
| | P K F Q | |
| | S | |
| | 3x 2x 2x 2 | = 24 Sequences |

### High Affinity Toxins Specific to Anopheles stefensi are Identified

Mesocyclops longisetus are Diptera reported to prey on mosquitoes, such as Anopheles stefensi, which are also Diptera. The use of M. longisetus in the control of mosquito populations is increasing, however, despite treatment with species such as M. longisetus, a need remains for toxins useful in the control of mosquitoes.

The library array of w-ACTX-1 peptides previously described is screened for specific binding to an Anopheles stefensi receptor. The library array is tested for specific binding to a M. longisetus receptor. One or more peptides showing specific binding to a A. stefensi receptor and demonstrating reduced binding, or no binding, to a M. longisetus receptor are selected. Peptides isolated according to this method may be used in mosquito control without a substantial adverse effect on natural predators such as M. longisetus.

Using a library prepared according to the invention facilitates identification of the target receptor, as each sequence represents one or more amino acid possibilities, which have been subject to natural selection for binding to receptors in various insects. Thus, while mosquito larvae may not express a receptor for which the aligned peptides have been selected to bind, all possible allowed substitutions are represented in the library, increasing the likelihood of identifying the desired binding.

As will be recognized by a person skilled in the art, these methods and principles may be applied to other sequences and/or organisms.

### EXAMPLE VII

### Phylogenetically Related μ-Conotoxins

Phylogenetically related μ-conotoxins are aligned as illustrated in Table 4.

TABLE 4
illustrating aligned μ-conotoxins:

| | | | |
|---|---|---|---|
| Se- | | | |
| quence | | | |
| Name | Species | Prey | Sequence |

| | | | |
|---|---|---|---|
| A3.3 | aurisiacus | Fish | ZKCCTGKKGSCSGKACKNLKCCS |
| | | | (SEQ ID NO: 17) |
| NB3.2 | nobilis | Mollusc | ZKCCTGKKGSCSGKACKNLKCCS |
| | | | (SEQ ID NO: 18) |
| A3.5 | aurisiacus | Fish | ZKCCTGRKGSCSGKACKNLKCCS |
| | | | (SEQ ID NO: 19) |
| Sm3.1 | stercusmus- | Fish | ZKCCTGKKGSCSGKACKNLKCCS |
| (NS) | carum | | (SEQ ID NO: 20) |
| Cn3.4 | consors | Fish | ZRCCTGKKGSCSGKACKSLKCCS |
| | | | (SEQ ID NO: 21) |
| 1m3.1 | imperialis | Worm | -RCCTGKKGSCSGKACKNLKCCA |
| | | | (SEQ ID NO: 22) |

TABLE 4.1

| Position | 1 5 10 15 20 | |
|---|---|---|
| | XXCCTGXKGSCSGKACKXLKCCX | SEQ ID NO: 23 |
| Allowed Sub- | | |
| stitutions at | | |
| Each | | |
| Position: | | |
| | ZK R N S | |
| | −R K S A | |
| | Z2 x2 x2 x2 | = 16 Sequen- |
| | | ces having a |
| | | Z at “posi- |
| | | tion 1.” |
| | −2 x2 x2 x2 | = 16 Sequen- |
| | | ces lacking |
| | | “position 1.” |

Table 4 illustrate the alignment of phylogenetically related sequences from the μ-conotoxin family. Conotoxins Cn3.4 and Im3.1 represent novel members of the μ-conotoxin family, the function and attributes of which are described in U.S. patent application Ser. No. 09/910,009, filed Jul. 23, 2001, the entirety of which is incorporated by reference. Table 4.1 illustrates the observed amino acids at each position (e.g., conserved amino acids and allowed substitutions). This Example illustrates an alignment having a deletion or insertion (indel) at a non-conserved position, wherein the indel is an allowed substitution. Position 1 in Table 4 and 4.1 can be viewed as an insertion in A3.3, Nb3.2, A3.5, Sm3.1 and Cn3.4 or a deletion in Im3.1. Therefore, an indel, which is used herein to describe the absence of an amino acid or nucleic acid, and a pyroglutamic acid (Z) are observed at position 1.

To generate a library having a deletion, the deleted position is treated individually. Thus, a first subset of sequences is generated wherein the first position is considered to be absent from all sequences (a non-position). This results in the production of a subset of 16 peptide sequences, 22 amino acids in length. A second subset is generated wherein the first position is pyroglutamic acid and the subset has 16 peptide sequences of 23 amino acids. The two subsets are then combined to generate the final set.

### EXAMPLE VIII

### Treatment of Conservative Substitutions

Table 5 illustrates the observed amino acids at each position (e.g., conserved and non-conserved amino acids) of phylogenetically related conotoxin sequences. Positions 8 and 14 illustrated in Table 5 may be considered to have conservative substitutions. Generating a library having a combination of all allowed substitutions, without considering conservative substitutions, would require the production of 864 peptides, as shown in SEQ ID NO:24. As the number of peptides necessary to produce a library increases, practical limitations (particularly for in vitro protein synthesis) become increasingly important. Therefore, where the number of peptides is prohibitively high or other considerations warrant a reduction in the complexity of the library, conservative substitutions may be used to achieve a further reduction in the complexity. Conserved substitutions, such as hydrophobic side groups may be treated as being equivalent.

Conservative substitutions are known in the art and may be determined, for example, by The PAM 250 matrix, originally created by Margaret Dayhoff, shown below.

PAM 250 Amino Acid Similarity Matrix

| | | | | | | | | | | | | | | | | | | | | |
|---|---|---|---|---|---|---|---|---|---|---|---|---|---|---|---|---|---|---|---|---|
| C | 12 | | | | | | | | | | | | | | | | | | | |
| G | −3 | 5 | | | | | | | | | | | | | | | | | | |
| P | −3 | −1 | 6 | | | | | | | | | | | | | | | | | |
| S | 0 | 1 | 1 | 1 | | | | | | | | | | | | | | | | |
| A | −2 | 1 | 1 | 1 | 2 | | | | | | | | | | | | | | | |
| T | −2 | 0 | 0 | 1 | 1 | 3 | | | | | | | | | | | | | | |
| D | −5 | 1 | −1 | 0 | 0 | 0 | 4 | | | | | | | | | | | | | |
| E | −5 | 0 | −1 | 0 | 0 | 0 | 3 | 4 | | | | | | | | | | | | |
| N | −4 | 0 | −1 | 1 | 0 | 0 | 2 | 1 | 2 | | | | | | | | | | | |
| Q | −5 | −1 | 0 | −1 | 0 | −1 | 2 | 2 | 1 | 4 | | | | | | | | | | |
| H | −3 | −2 | 0 | −1 | −1 | −1 | 1 | 1 | 2 | 3 | 6 | | | | | | | | | |
| K | −5 | −2 | −1 | 0 | −1 | 0 | 0 | 0 | 1 | 1 | 0 | 5 | | | | | | | | |
| R | −4 | −3 | 0 | 0 | −2 | −1 | −1 | −1 | 0 | 1 | 2 | 3 | 6 | | | | | | | |
| V | −2 | −1 | −1 | −1 | 0 | 0 | −2 | −2 | −2 | −2 | −2 | −2 | −2 | 4 | | | | | | |
| M | −5 | −3 | −2 | −2 | −1 | −1 | −3 | −2 | 0 | −1 | −2 | 0 | 0 | 2 | 6 | | | | | |
| I | −2 | −3 | −2 | −1 | −1 | 0 | −2 | −2 | −2 | −2 | −2 | −2 | −2 | 4 | 2 | 5 | | | | |
| L | −6 | −4 | −3 | −3 | −2 | −2 | −4 | −3 | −3 | −2 | −2 | −3 | −3 | 2 | 4 | 2 | 6 | | | |
| F | −4 | −5 | −5 | −3 | −4 | −3 | −6 | −5 | −4 | −5 | −2 | −5 | −4 | −1 | 0 | 1 | 2 | 9 | | |
| Y | 0 | −5 | −5 | −3 | −3 | −3 | −4 | −4 | −2 | −4 | 0 | −4 | −5 | −2 | −2 | −1 | −1 | 7 | 10 | |
| W | −8 | −7 | −6 | −2 | −6 | −5 | −7 | −7 | −4 | −5 | −3 | −3 | 2 | −6 | −4 | −5 | −2 | 0 | 0 | 17 |
| | C | G | P | S | A | T | D | E | N | Q | H | K | R | V | M | I | L | F | Y | W |

The PAM 250 matrix above has been arranged so that similar amino acids are close to each other. As illustrated in the above Matrix, Ala and Ser or Leu and Met are conservative substitutions. Therefore, the observed substitution of Ala and Ser may be removed from the determination of allowed substitutions by assuming that Ala and Ser are equivalent, as in SEQ ID NO:27.

TABLE 5

| | |
|---|---|
| 1 5 10 15 20 | |
| NKCCGXXXXCPKYXRXXXICSCC | (SEQ ID NO: 24) |
| KDAD S DRL | |
| PGSS F NNM | |
| E W Y | |
| 3222 3 223 | = 864 (Conserved amino |
| | acids not considered) |
| KDSD S DRM | A is assumed to be equiva- |
| PG S F NNY | lent to S; L is assumed to |
| E W | be equivalent to M. |
| NKCCGXXXXCPKYXRXXXICSCC | (SEQ ID NO: 25) |
| 32 2 3 222 | = 288 |

Although the number of peptides required in SEQ ID NO:24 is probably not sufficiently large to pose a problem, it serves as an example of how the number of representative peptides may be reduced.

By limiting either positions 2 and/or 7 to one of the allowed conservative substitutions, the ultimate number of peptides required is reduced. For example, utilizing the conservative amino acids observed at positions 2 and 7, the number of peptides required is reduced from 864 to 288.

### EXAMPLE IX

### Indels and Conservative Substitutions

Table 6 illustrate an alignment of phylogenetically related sequences. The observed amino acids at each position are shown in Table 6.1. This Example illustrates an alignment having multiple indels and conservative substitutions.

To synthesize a peptide library having an indel, the indel position is treated individually for the purposes of chemical synthesis of the library. The peptide subsets are then combined to generate the final set or library, as illustrated in Table 6.2.

TABLE 6

illustrating aligned β-conotoxins:

| | | | |
|---|---|---|---|
| Sequence | | | |
| Name | Species | Prey | Sequence |

| | | | | |
|---|---|---|---|---|
| Wil4.1 | Wittigi | Fish | SSDGSDPKAKKQCMWKRCIPDQSR---L-EEDE | (SEQ ID NO: 26) |
| Cil4.4 | cinereus | Mollusc | SSDG---KAKKQCAWKTCVPTQWRRRDLKEKDE | (SEQ ID NO: 27) |
| Cil4.3 | cinereus | Fish | SSDG---KAKRNCFWKACVPEQWRQRDPKEKDE | (SEQ ID NO: 28) |
| Cil4.2 | cinereus | Fish | SSDG---KAKRNCFWKACVPEQWRQRDLKELDE | (SEQ ID NO: 29) |

TABLE 6.1

| Position | 1 5 10 15 20 25 30 | |
|---|---|---|
| | SSDGXXXKAKXXCXWKXCXPXQXRXXXXXEKDE | SEQ ID NO: 30 |
| Allowed Substitutions | | |
| at Each Position: | | |
| | SDP KQ M R I D S ---L- | |
| | --- RN A T V T W RRDPK | |
| | F A E Q | |

TABLE 6.2

| | |
|---|---|
| Position | 1 5 10 15 20 25 30 |

| | | |
|---|---|---|
| | SSDGXXXKAKXXCXWKXCXPXQXRXXXXXEKDE | SEQ ID NO: 30 |
| | SSDG---KAKKQCMWKRCIPDQSRRRDPKEKDE | SEQ ID NO: 31 |
| | RN A T V T W Q | |
| | F A E | |
| Subset #1 | 22 3 3 2 3 2 2 | = 864 peptides |
| | SSDG---KAKXQCMWKRCIPDQSR---P-EKDE | SEQ ID NO: 32 |
| | RN A T V T W | |
| | F A E | |
| Subset #2 | 22 3 3 2 3 2 | = 432 peptides |
| | SSDGSDPKAKKQCMWKRCIPDQSRRRDPKEKDE | SEQ ID NO: 33 |
| | RN A T V T W Q | |
| | F A E | |
| Subset #3 | 22 3 3 2 3 2 2 | = 864 peptides |
| | SSDGSDPKAKKQCMWKRCIPDQSR---P-EKDE | SEQ ID NO: 34 |
| | RN A T V T W | |
| | F A E | |
| Subset #4 | 22 3 3 2 3 2 | = 432 peptides |

Subsets 1 to 4, shown in Table 6.2, are combined to produce a final set or library having 2,592 unique peptides. Alternatively, conservative substitutions can be taken into account to reduce the number of peptides in the library. Table 6.3 illustrates the positions having conservative substitutions (the representative amino acid is shown in outline text) and the effect of treating conservative substitutions as equivalent amino acids.

TABLE 6.3

| | |
|---|---|
| Position | 1 5 10 15 20 25 30 |

| | | |
|---|---|---|
| | SSDGXXXKAKXXCXWKXCXPXQXRXXXXXEKDE | SEQ ID NO: 30 |
| | SSDG---KAKKQCMWKRCIPDQSRRRDPKEKDE | SEQ ID NO: 35 |
| | A T V T W Q | K and R treated as K |
| | F A E | Q and N treated as Q |
| Subset #1A | 3 3 2 3 2 2 | = 216 peptides |
| | SSDG---KAKKQCMWKRCIPDQSR---P-EKDE | SEQ ID NO: 36 |
| | A T V T W | |
| | F A E | |
| Subset #2A | 3 3 2 3 2 | = 108 peptides |
| | SSDGSDPKAKKQCMWKRCIPDQSRRRDPKEKDE | SEQ ID NO: 37 |
| | A T V T W Q | |
| | F A E | |
| Subset #3A | 3 3 2 3 2 2 | = 216 peptides |
| | SSDGSDPKAKKQCMWKRCIPDQSR---P-EKDE | SEQ ID NO: 38 |
| | A T V T W | |
| | F A E | |
| Subset #4A | 3 3 2 3 2 | = 108 peptides |

Subsets 1A to 4A, shown in Table 6.3, are combined to produce a final set or library having 648 peptides. Treatment of conservative substitutions as functionally equivalent reduces the number of required peptides from 2,592 to 648.

### EXAMPLE X

### Computation of Sequences

Sequences are entered into a computer program, for example, Mn3.1, Ac3.3, A3.1, M3.7, M3.3, Cn3.1, A3.3, Nb3.2, A3.5, Sm3.1 and Cn3.4, which compares the relationship of the sequences and, preferably, generates a visual output, such as a phylogenetic tree. The operator may determine the desired phylogenetically related sequences and input the sequence identifications to the computer. Alternatively, the desired degree of phylogenetic relation may be set at the onset of analysis and the output of selected phylogenetically related sequences may be automatically routed for further analysis of allowed substitutions and optionally conservative substitutions. In either case, the computer program subsequently identifies the observed members at each position as described herein. The sequences required for generation of a set of sequences composed of the union of observed members at each position are then output to the end user.

The invention may be implemented in computer programs executing on computers, having at least one processor, at least one data storage system (including volatile and non-volatile memory and/or storage elements), at least one input device, and at least one output device. Program code is applied to input data to perform the functions described herein and generate output information. The output information is applied to one or more output devices, in known fashion.

The program may be implemented in a high level procedural or object oriented programming language, so as to communicate with a computer system. The program may also be implemented in assembly or machine language, if desired. The language may be any language capable of being compiled and/or interpreted by a computer.

A computer program may be stored on any storage media or device (e.g., ROM or magnetic diskette) readable by a general or special purpose computer, for configuring and operating the computer when the storage media or device is read by the computer to perform the procedures described herein. The invention may also be implemented as a computer-readable storage medium, configured with a computer program, where the storage medium so configured causes a computer to operate in a specific and predefined manner to perform the functions described herein.

The output to the end user, may, where appropriate and desirable, be used as the input for an automated peptide or nucleic acid synthesizer. The coupling reactions can be performed automatically, as on a Beckman 990 automatic synthesizer, using a program such as that reported in Rivier et al. 1978. The input may also, where appropriate and desirable, constitute the output of an automated sequencer.

### EXAMPLE XI

### Nucleic Acids

Table 7 shows the alignment of phylogenetically related nucleic acid sequences. The sequences shown are reported TATA-box sites in Saccharomyces cerevisiae. As illustrated in Table 7, positions five to nine exhibit different allowed substitutions (shown in bold).

TABLE 7

| | |
|---|---|
| Accession Number | Sequence |

| | |
|---|---|
| X04493 | tatatataa |
| M22785 | tatataa |
| X15154 | tatataa |
| E01242 | tatataa |
| E00298 | tataata |
| X03372 | tataaaag |
| X02649 | tataaa |
| X56886 | tataaa |
| Conserved and Non-conserved | tataxxxxx |
| positions | |
| Allowed Substitutions | tataa |
| | atag |
| | --- |

Positions one to four are conserved positions and positions five to nine are non-conserved. The allowed substitutions at positions five and six are each A or T. The allowed substitutions at positions seven and eight are A, T or an indel and A, G or an indel, respectively. The allowed substitutions at position nine are an A or an indel. Thus, the first four positions are invariant and the last five positions contain allowed substitutions.

A library of nucleic acid sequences may be generated by limiting positions five through nine to the allowed substitutions. For example, a first subset of sequences is generated having a length of six nucleotides. The sequences are: tatata, tatatt, tataaa, tataat. A second subset of sequences is generated having a length of seven nucleotides. The sequences are: tatatta, tatattt, tatataa, tatatat, tataata, tataatt, tataaaa, tataata. A third subset of sequences is generated having a length of eight nucleotides. The sequences are: tatataaa, tatataag, tatatata, tatatatg, tatattaa, tatattag, tatattta, tatatttg, tataaaaa, tataaaag, tataaata, tataaatg, tataataa, tataatag, tataatta, tataattg. A fourth subset of sequences is generated having a length of nine nucleotides. The sequences are: tatataaaa, iatataaga, tatatataa, tatatatga, tatattaaa, tatattaga, tatatttaa, tatatttga, tataaaaaa, tataaaaga, tataaataa, tataaatga, tataataaa, tataataga, tataattaa, tataattga. In this example, the four subsets of sequences are combined to form the set of sequences composed of the union of observed nucleotides or indels at each position.

The library may be screened to identify a TATA-box sequence having a desired property. For example, a TATA-box binding protein may be synthesized and attached to a column. The library may then be passed over the column under conditions favorable for binding of the TATA-box binding protein to members of the library. Non-binding members may be removed in the flow through and subsequent washing steps. Bound members may be eluted and reapplied to the column. These steps may be repeated as often as appropriate and desirable. Following a final washing step the bound sequences are eluted and directly sequenced or cloned into vectors known in the art. By this procedure optimal binding sites for the TATA-box binding protein are isolated.

The conotoxin Cn3.4 is a novel member of the μ-conotoxin family, having the following sequence: XXCCXGXXGXCXGXACXXXXCCX (SEQ ID NO:39). The conotoxin Im3.1 is a novel member of the μ-conotoxin family, having the following sequence: XCCXGXXGXCXGXACXNXXCCA (SEQ ID NO:40). The venum ducts from the Conus genus express an active toxin, for example, Cn3.4, wherein the amino acid sequence may contain either pyroglutamate or glutamine and retain biological activity.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### References

All references, including publications, patents, and patent applications, cited herein are hereby incorporated by reference to the same extent as if each reference were individually and specifically indicated to be incorporated by reference and were set forth in its entirety herein.

Altschul et al. (1990), Basic local alignment search tool. J. Mol. Biol., 215:403-410.

Altschul et al. (1997) Gapped BLAST and PSI-BLAST: A New Generation of Protein Database Search Programs, Nucl. Acids Res., 25:3389-3402

Bonaldo et al. (1996) Normalization and Subtraction: Two Approaches to Facilitate Gene Discovery, Genome Res. 6:791-806.

Bowersox, S. S., and R. Luther (1998) Pharmacotherapeutic potential of omega-conotoxin MVIIA (SNX-111), an N-type neuronal calcium channel blocker found in the venom of Conus magus, Toxicon 36:1651-1658.

Castelloe and Templeton (1994) Root Probabilities for Intraspecific Gene Trees Under Neutral Coalescent Theory, Mol. Phylogenet. Evol., 3:102-113.

Catterall, W. A. (1980) Neurotoxins that act on voltage-sensitive sodium channels in excitable membranes, Annu. Rev. Pharmacol. Toxicol. 20:15-43.

COMBINATORIAL PEPTIDE AND NONPEPTIDE LIBRARIES: A HANDBOOK (Günther Jung ed., 1996).

Conticello et al., (2001) Mechanisms for Evolving Hypervariability: The Case of Conopeptides, Mol. Biol. Evol. 18(2):120-131.

Cruz, L. J., and Olivera, B. M. (1986) Calcium channel antagonists. Omega-conotoxin defines a new high affinity site, J. Biol. Chem. 261(14):6230-6233.

Current Protocols in Molecular Biology (F. M. Ausubel et al., eds., 1987) Supplement 30, section 7.718, Table 7.71.

Devereux, J., et al., (1984) A Comprehensive Set of Sequence Analysis Programs for the VAX, Nucl. Acids Res., 12(1):387-395.

Doray et al. (2002) Autoinhibition of the ligand-binding site of GGA1/3 VHS domains by an internal acidic cluster-dileucine motif, Proc. Natl. Acad. Sci. USA, 99(12):8072-8077.

Drakopoulou et al., (1998), Consequence of the Removal of Evolutionary Conserved Disulfide Bridges on the Structure and Function of Charybdotoxin and Evidence that Particular Cysteine Spacings Govern Specific Disulfide Bond Formation, Biochemistry 37(5):1292-1301.

Duda, T. F., and S. R. Palumbi (1999) Molecular Genetics of Ecological Diversification: Duplication and Rapid Evolution of Toxin Genes of the Venomous Gastropod Conus. Proc. Natl. Acad. Sci. USA, 96:6820-6823.

Felsenstein, J. 1989. PHYLIP—Phylogeny Inference Package (Version 3.2), Cladistics 5: 164-166

Fletcher, J. I., et al. (1997) The Structure of a Novel Insecticidal Neurotoxin, Omega-atracotoxin-HV1, from the Venom of an Australian Funnel Web Spider, Nat Struct Biol. 4(7):559-66.

GEORG THIEME VERLAG, STUTTGART, W. GER, METHODEN DER ORGANISCHEN CHEMIE (HOUBEN-WEYL): SYNTHESE VON PEPTIDEN, (E. Wunsch ed. 1974).

Higgins et al. (1992) CLUSTAL V: Improved Software For Multiple Sequence Alignment, Comput Appl Biosci 8:189-191.

Hillyard, D. R., et al., (1992) A New Conus Peptide Ligand For Mammalian Presynaptic Ca2+ Channels, Neuron 9:69-77.

Hlavacek and Ragnarsson (2001) Solid Phase Synthesis of Partially Protected Tocinoic Acid: Optimization with Respect to Resin and Protecting Groups, J Pept. Sci. 7(7):349-57.

Jayawickreme et al. (1994) Creation and Functional Screening of a Multi-use Peptide Library, Proc. Natl. Acad. Sci. USA 91:1614-1618.

Jones et al. (2001) Composition and Therapuetic utility of conotoxins from genus Conus. Patent Status 1996-2000, Exp. Opin. Ther. Patents, 11(4):603-623.

Kohn, A. J., and J. W. Nybakken (1975) Ecology of Conus on Eastern Indian Ocean Fringing Reefs: Diversity of Species and Resource Utilization, Mar. Biol., 29:211-234.

Kumar et al. (1993) MEGA: Molecular Evolutionary Genetics Analysis, version 1.01, The Pennsylvania State University, University Park, Pa. 16802.

Li (1993) Unbiased Estimation of the Rates of Synonymous and Nonsynonymous Substitution, J. Mol. Evol., 36:96-99.

Li et al. (1985) A new Method for Estimating Synonymous and Nonsynonymous Rates of Nucleotide Substitution Considering the Relative Likelihood of Nucleotide and Codon Changes, Mol. Biol. Evol., 2:150-174.

Luo et al., (1990) Single-residue Alteration in ω-conotoxin PnIA switches its nAChR subtype selectivity, Biochem., 38:14542.

McIntosh et al., (1999) Conus Peptides as Probes for Ion Channels, Methods Enzymol. 294:605-24.

McIntosh, J. M. (2000) Isolation And Characterization Of A Novel Conus Peptide With Apparent Antinociceptive Activity, J. Biol. Chem. 275(42):32391-32397.

Messier and Stewart (1997) Episodic Adaptive Evolution Of Primate Lysozymes, Nature 385:151-154.

McBride, J. D. et al. (1996) Selection of Chymotrypsin Inhibitors from a Conformationally-constrained Combinatorial Peptide Library, J. Mol. Biol. 259:819-827.

Nei and Gojobori (1986) Simple Methods for Estimating the Numbers of Synonymous and Nonsynonymous Nucleotide Substitutions, Mol. Biol. Evol., 3:418-426.

NEI, MOLECULAR EVOLUTIONARY GENETICS (New York, Columbia University Press 1987).

Norton and Pallaghy (1998), The Cystine Knot Structure of Ion Channel Toxins and Related Polypeptides, Toxicon 36(11):1573-83.

Ohno et al. (1998) Molecular Evolution of Snake Toxins: is the Functional Diversity of Snake Toxins Associated with a Mechanism of Accelerated Evolution? Prog. Nucleic Acid Res. Mol. Biol., 59:307-364.

Olivera, B. M. et al. (1996). U.S. Pat. No. 5,514,774.

Pabreza et al. (1991) ³H Cytisine Binding To Nicotinic Cholinergic Receptors In Brain, Mol. Pharmacol. 39:9-12.

Page (1996) TreeView: An Application to Display Phylogenetic Trees on Personal Computers, Comput. Appl. Biosci., 12:357-358.

Rivier et al. Biopolymers, 1978, 17, pp 1927-1938.

Saitou and Nei (1987) The Neighbor-joining Method: a New Method for Reconstructing Phylogenetic Trees, Mol. Biol. Evol., 4:406-425.

Sasaki et al. (1999) Synthesis, Bioactivity and Cloning of the L-type Calcium Channel Blocker ω-Conotoxin TxVII, Biochemistry 38:12876-12884.

SCHRODER & LUBKE, THE PEPTIDES, 1 pp 72-75, Academic Press (1965).

Solid-Phase Peptide Synthesis, (Stewart & Young, Freeman & Co. ed. 1969).

Stoilov et al. (2002), YTH: A New Domain In Nuclear Proteins, Trends Biochem. Sci. 27(10):495-497.

Swofford, D. L. (1993). PAUP: Phylogenetic Analysis Using Parsimony Version 1.3.1, Illinois Natural History Survey, Illinois.

Swofford, D. L. (1998). PAUP*. Phylogenetic Analysis Using Parsimony (*and other methods) Version 4, Sinauer, Sunderland, Mass.

Tedford, H. W., et al. (2001) Functional Significance Of The Beta Hairpin In The Insecticidal Neurotoxin Omega-Atracotoxin-Hv1a. J. Biol. Chem. 276:26568-26576.

Templeton (1996) Contingency Tests of Neutrality using Intra/Interspecific Gene Trees: The Rejection of Neutrality for the Evolution of the Mitochondrial Cytochrome Oxidase II Gene in the Hominoid Primates, Genetics 144:1263-1270.

Wang, X.-H et al. (1999) Structure-Function Studies Of ω-Atracotoxin, A Potent Antagonist Of Insect Voltage-Gated Calcium Channels, European J. Biochemistry 264:488-494.

Wells et al. (1987) Recruitment of Substrate-specifity Properties from One Enzyme into a Related one by Protein Engineering. Proc. Natl. Acad. Sci. USA 84: 5167-71.

WEN-HSIUNG LI, MOLECULAR EVOLUTION pp. 99-176, 122 (1997).

Wold, F. (1981) In vivo Chemical Modification of Proteins (Post-translational Modification), Annu. Rev. Biochem., 50:783-814.

We claim:

1. A method of generating a set of possible amino acid sequence combinations, said method comprising:
a) selecting phylogenetically related amino acid sequences;
b) aligning said phylogenetically related amino acid sequences to create an alignment;
c) identifying observed amino acid residues or indels occupying each position in said alignment of said phylogenetically related sequences; and
d) generating a set of possible amino acid sequence combinations consisting essentially of the union of the observed amino acid residues or indels identified at each position.

2. The method according to claim 1, further comprising:
screening or selecting said set of possible amino acid sequence combinations to identify ligand binding pair members; and
isolating an identified individual ligand binding pair member or a mixed population of identified individual ligand binding pair members.

3. An identified individual ligand binding member produced by the method of claim 2.

4. The method according to claim 1, wherein said phylogenetically related sequences are from the same genus.

5. The method according to claim 4, wherein said genus is Conus or Hadronyche.

6. The method according to claim 5, wherein said phylogenetically related sequences are conotoxins.

7. The method according to claim 6, wherein said conotoxins are mature toxins.

8. The method according to claim 1, wherein said phylogenetically related sequences comprise a clade.

9. The method according to claim 1, wherein said method is performed at least in part by a computer.

10. The method according to claim 1, wherein each member of said set of possible amino acid sequence combinations occupies an isolated location on a physical support.

11. The method according to claim 2, wherein each member of said peptide library occupies an isolated location on a physical support.

12. The method according to claim 2, wherein an identified individual ligand binding pair member derived from a screened or selected set of possible amino acid sequence combinations is produced.

13. The method according to claim 2, wherein an identified individual ligand binding pair member derived from a screened or selected set of possible amino acid sequence combinations is produced in a recombinant host organism.

14. The method according to claim 1, further comprising:
(e) identifying at least one position having amino acids or indels which are conservative changes in said alignment;
(f) selecting a representative from said conservative changes; and
(g) treating all said conservative changes identified in (e) as equivalent to said representative from (f) at said position.

15. The method according to claim 13, wherein said representative is selected by frequency of occurrence in said phylogenetically related sequences.

16. The method according to claim 13, wherein each member of said set of possible amino acid sequence combinations occupies an isolated location on a physical support.

17. The method according to claim 14, further comprising:
screening or selecting said set of possible amino acid sequence combinations to identify ligand binding pair members;
isolating an identified individual ligand binding pair member or a mixed population of identified individual ligand binding pair members.

18. An individual ligand binding member produced by the method of claim 17.

19. A method of generating a set of possible amino acid sequence combinations comprising:
selecting phylogenetically related amino acid sequences;
aligning said phylogenetically related amino acid sequences to create an alignment;
identifying observed amino acid residues or indels occupying each position in said alignment of said phylogenetically related sequences;
generating a set of possible amino acid sequence combinations consisting essentially of the union of the observed amino acid residues or indels identified at each position;
generating nucleic acids encoding a set of fusion proteins, wherein said each fusion protein of said set comprises a carrier protein and a peptide, wherein said peptide consists essentially of said set of possible amino acid sequence combinations;
introducing said nucleic acids encoding said set of fusion proteins into a DNA vector, thereby producing a set of recombinant DNA vectors;
transforming host cells with said set of recombinant DNA vectors; and
culturing said transformed host cells under conditions suitable for expression of said fusion proteins.

20. The method according to claim 19, further comprising:
screening or selecting said expressed fusion proteins to identify ligand binding pair members; and
isolating an identified individual ligand binding pair member or a mixed population of identified individual ligand binding pair members.

21. An individual ligand binding member produced by the method of claim 20.

22. The method according to claim 19, wherein said fusion is a tripartite fusion having a cleavage site between said carrier protein and said peptide.

23. A method of generating a set of possible amino acid sequence combinations, said method comprising:
selecting phylogenetically related nucleic acid sequences;
aligning said phylogenetically related nucleic acid sequences to create an alignment;
identifying observed nucleotides or indels occupying each position in said alignment of said phylogenetically related sequences; and
generating a set of possible nucleic acid sequence combinations consisting essentially of the union of the observed nucleotides or indels identified at each position.

24. The method according to claim 22, wherein said nucleic acid sequences encode polypeptides.

25. A computer-assisted method for generating a library of peptides, comprising:
receiving into a computer system a string describing at least two amino acid sequences;
executing an alignment algorithm in a computer system to compute an alignment score indicating the optimal alignment of said at least two amino acid sequences;
inputting into the computer system parameters defining a desired degree of relation to define phylogenetically related sequences;
generating, from the optimal alignment, a phylogenetic relationship between said at least two amino acid sequences;
identifying observed amino acid residue or indel occupying each position in said alignment of phylogenetically related sequences;
defining a set of possible amino acid sequence combinations consisting essentially of the union of the observed amino acid residues or indels indentified at each position; and
generating said set of possible amino acid sequence combinations to form a library of peptides.

26. A set of amino acid sequence combinations produced by a process comprising:
selecting phylogenetically related amino acid sequences;
aligning said phylogenetically related amino acid sequences to create an alignment;
identifying observed amino acid residues or indels occupying each position in said alignment of said phylogenetically related sequences; and
generating a set of possible amino acid sequence combinations consisting essentially of the union of the observed amino acid residues or indels identified at each position.

27. The set of amino acid sequence combinations of claim 26, wherein said phylogenetically related sequences are from the same genus.

28. The set of amino acid sequence combinations of claim 27, wherein said genus is Conus.

29. The set of amino acid sequence combinations of claim 28, wherein said phylogenetically related sequences are conopeptides.

30. The set of amino acid sequence combinations of claim 29, wherein said conopeptides are mature toxins.

31. The set of amino acid sequence combinations of claim 26, wherein each member of said set of amino acid sequences occupies an isolated location on a physical support.

32. A peptide of claim 26, the process further comprising:
screening or selecting said set of amino acid sequence combinations for a desired property;
identifying one or more individual ligand binding peptide from said set of amino acid sequence combinations; and
isolating said individual ligand binding peptide or a mixed population of said individual ligand binding peptides.

33. The peptide of claim 32, wherein said desired property is binding to a receptor.

34. A set of peptides, comprising:
a combination of peptides consisting essentially of a union of observed members at each position.

35. The set of peptides of claim 34, wherein said union of observed members comprises conopeptides.

36. The set of peptides of claim 35, wherein said union of observed members comprises mature conopeptides.

37. The set of peptides of claim 36, wherein said combination of peptides is attached to a physical support.

38. The set of peptides of claim 34, wherein said combination of peptides is encoded by at least one nucleic acid sequence.

39. The set of peptides of claim 34, wherein said combination of peptides is expressed in a host cell line.

40. An isolated conotoxin peptide comprising the conotoxin set forth in SEQ ID NO:39 and derivatives thereof.

41. An isolated conotoxin peptide comprising the conotoxin set forth in SEQ ID NO:40 and derivatives thereof.


SEQUENCE LISTING (40 sequences)

SEQ ID NO: 1 - protein, 18 aa, Conus betulinus
GGCCSHPACAVNHPELCG

SEQ ID NO: 2 - protein, 18 aa, Conus ammiralis
QECCSYPACNLDHPELCG

SEQ ID NO: 3 - protein, 18 aa, Conus textile
PQCCSHPACNVDHPEICR

SEQ ID NO: 4 - protein, 18 aa, Conus radiatus
PQCCSHPACNVDHPEICD

SEQ ID NO: 5 - protein, 18 aa, Conus aureus
PECCSHPACNVSHPEICR

SEQ ID NO: 6 - protein, 18 aa, Conus radiatus
PECCSHPACNVDHPEICR

SEQ ID NO: 7 - protein, 18 aa, Artificial
  subdivision of phylogenetically related sequences alpha-conotoxins showing conserved and non- conserved positions.
XXCCSXPACXXXHPEXCX

SEQ ID NO: 8 - RNA, 54 nt, Artificial
  Artificial sequence encoding the alpha- conotoxin library.
nnannaugcuguuccnaccccgccugcnncnucnnccaccccgagnuaugcnnn

SEQ ID NO: 9 - protein, 37 aa, Hadronyche versuta
SAVCIPSGQPCPYSKYCCSGSCTYKTNENGNSVQRCD

SEQ ID NO: 10 - protein, 37 aa, Hadronyche versuta
SPTCIPSGQPCPYNENCCSQSCTYKENENGNTVKRCD

SEQ ID NO: 11 - protein, 37 aa, Hadronyche versuta
SPTCIPSGQPCPYNENCCSKSCTYKENENGNTVQRCD

SEQ ID NO: 12 - protein, 37 aa, Hadronyche versuta
SSTCIPSGQPCPYNENCCSQSCTFKENENGNTVKRCD

SEQ ID NO: 13 - protein, 37 aa, Hadronyche versuta
SSTCIPSGQPCPYNENCCSQSCTYKENENGNTVKRCD

SEQ ID NO: 14 - protein, 37 aa, Hadronyche versuta
SPTCIPSGQPCPYNENCCSQSCTFKENENGNTVKRCD

SEQ ID NO: 15 - protein, 37 aa, Artificial
  Conserved and non-conserved positions in Hadronyche versuta w-ACTX-1 sequences
SXXCIPSGGPCPYXXXCCSXSCTXKXNENGNXVXRCD

SEQ ID NO: 16 - protein, 37 aa, Artificial
  Library for w-ACTX-1 excluding P81893 from subdivision of phylogenetically related sequences.
SXTCIPSGGPCPYNENCCSXSCTXKENENGNTVXRCD

SEQ ID NO: 17 - protein, 23 aa, Conus aurisiacus
  MOD_RES (1)..(1): Z is pyrrolidone carboxylic acid or pyroglutamate
ZKCCTGKKGSCSGKACKNLKCCS

SEQ ID NO: 18 - protein, 23 aa, Conus nobilis
  MOD_RES (1)..(1): Z is pyrrolidone carboxylic acid or pyroglutamate
ZKCCTGKKGSCSGKACKNLKCCS

SEQ ID NO: 19 - protein, 23 aa, Conus aurisiacus
  MOD_RES (1)..(1): Z is pyrrolidone carboxylic acid or pyroglutamate
ZKCCTGRKGSCSGKACKNLKCCS

SEQ ID NO: 20 - protein, 23 aa, Conus stercusmuscarum
  MOD_RES (1)..(1): Z is pyrrolidone carboxylic acid or pyroglutamate
ZKCCTGKKGSCSGKACKNLKCCS

SEQ ID NO: 21 - protein, 23 aa, Conus consors
  MOD_RES (1)..(1): Z is pyrrolidone carboxylic acid or pyroglutamate
ZRCCTGKKGSCSGKACKSLKCCS

SEQ ID NO: 22 - protein, 22 aa, Conus imperialis
RCCTGKKGSCSGKACKNLKCCA

SEQ ID NO: 23 - protein, 23 aa, Artificial
  ?-Conotoxins Consensus Sequence
XXCCTGXKGSCSGKACKXLKCCX

SEQ ID NO: 24 - protein, 23 aa, Artificial
  Conserved and nonconserved sequence derived from alignment.
NKCCGXXXXCPKYXRXXXICSCC

SEQ ID NO: 25 - protein, 23 aa, Artificial
  Conserved and nonconserved sequence derived from alignment where conservative substitutions are assigned a single representative
NKCCGXXXXCPKYXRXXXICSCC

SEQ ID NO: 26 - protein, 29 aa, Conus wittigi
SSDGSDPKAKKQCMWKRCIPDQSRLEEDE

SEQ ID NO: 27 - protein, 30 aa, Conus cinereus
SSDGKAKKQCAWKTCVPTQWRRRDLKEKDE

SEQ ID NO: 28 - protein, 30 aa, Conus cinereus
SSDGKAKRNCFWKACVPEQWRQRDPKEKDE

SEQ ID NO: 29 - protein, 30 aa, Conus cinereus
SSDGKAKRNCFWKACVPEQWRQRDLKELDE

SEQ ID NO: 30 - protein, 33 aa, Artificial
  Observed amino acids at each position.
SSDGXXXKAKXXCXWKXCXPXQXRXXXXXEKDE

SEQ ID NO: 31 - protein, 33 aa, Artificial; identical to SEQ ID NO: 30
  Subset 1

SEQ ID NO: 32 - protein, 33 aa, Artificial; identical to SEQ ID NO: 30
  Subset 2

SEQ ID NO: 33 - protein, 33 aa, Artificial; identical to SEQ ID NO: 30
  Subset 3

SEQ ID NO: 34 - protein, 33 aa, Artificial; identical to SEQ ID NO: 30
  Subset 4

SEQ ID NO: 35 - protein, 33 aa, Artificial; identical to SEQ ID NO: 30
  Subset 1A

SEQ ID NO: 36 - protein, 33 aa, Artificial; identical to SEQ ID NO: 30
  Subset 2A

SEQ ID NO: 37 - protein, 33 aa, Artificial; identical to SEQ ID NO: 30
  Subset 3A

SEQ ID NO: 38 - protein, 33 aa, Artificial; identical to SEQ ID NO: 30
  Subset 4A

SEQ ID NO: 39 - protein, 23 aa, Conus consors
  MISC_FEATURE (1)..(1): X is pyroglutamate or glutamine
XXCCXGXXGXCXGXACXXXXCCX

SEQ ID NO: 40 - protein, 22 aa, Conus imperialis
  MISC_FEATURE (1)..(1): X is Arginine or Lysine
XCCXGXXGXCXGXACXNXXCCA